SEQUENCE LISTING (4 sequences)

SEQ ID NO: 1 - DNA, 96 bp, Homo sapiens
ccatatcttcttaacaataatgtctgatccaagcactggcaaattgaagagagtctttccatgacattaatatatggatccaggaaagagaagatt

SEQ ID NO: 2 - DNA, 942 bp, Homo sapiens
ttcttttactttagaattgaaaaatgatagtggccctggaccatcagcagccatctttctcccctcatgaaaagagcacatccatggatttaacttaacattagaggaggtacaatgagaatatggaaatagagcctaaataattttattttgcccctagatctagcatatgagaagcaaatatgttctgcatttcccagtttataagccaataaattctcctttgtgcttaaagcattcgagttacattctgacacttgcaaccaagaatcctgacaaatacatctttggaatttgtatctcctctgaattacagtgactggtacataggactcaaaaatatgtacataggactggtacatagtacagtgactggtacataggacttaaaaatatgttacatggatgaatgaacagcttagcaaaaggtctgggaatgacctctgcgtaaagtgaatttctgctgcccccaaaactgcctagtgtgtgctgttcatcaacaatgcacccacttcttgcactttgatgtagagaggccataccacctgtgtctacatgtggttactacttaaacaatacttacatagttagcgagtggccaaaatgtctattaatctgtaaacacttggaaagaatccagtttgtattcttattttcctggtttaaagtgctaaatgaaccaaaagttagggaagctcttttcagaaaagccatcatgcctgatccaatccactgaacaaaagatttaggagctctccatctgatattagtctttggaatagccttgacttaagtgtgcttgagcctgtgactgtgggtgaaagtgtgtgagcgatgattttcttcttgcaggtaccaatcgggtaaattctcaagtgtgtagctactgaggactctgcctcaaatggaagaaggcagagcttccaaaccattagtaagtta

SEQ ID NO: 3 - DNA, 96 bp, Homo sapiens; identical to SEQ ID NO: 1

SEQ ID NO: 4 - DNA, 100 bp, Homo sapiens
cttcttgcaggtaccaatcgggtaaattctcaagtgtgtagctactgaggactctgcctcaaatggaagaaggcagagcttccaaaccattagtaagtta

(19) United States
(12) Patent Application Publication
(10) Pub. No.: US 2012/0283108 A1
(43) Pub. Date: Nov. 8, 2012

(54) METHOD FOR PHASED GENOTYPING OF A DIPLOID GENOME

(76) Inventor: Nicholas M. Sampas, Loveland, CO (US)

(21) Appl. No.: 13/462,645

(22) Filed: May 2, 2012

Related U.S. Application Data
(60) Provisional application No. 61/482,069, filed on May 3, 2011.

(51) Int. Cl. C12Q 1/68; C40B 30/00; G01N 21/64; C40B 20/00; C12M 1/34

(52) U.S. Cl. 506/2; 435/6.11; 435/287.2; 506/7

(57) ABSTRACT

A method of sample analysis is provided. In certain embodiments the method comprises: a) obtaining from a diploid individual a chromosomal sample that comprises maternally-derived chromosomes and homologous paternally-derived chromosomes; b) determining the parent of origin of a first chromosome of the sample by detecting a parent-specific copy number variation relative to a second chromosome that is homologous to the first chromosome; c) isolating the first chromosome; and d) genotyping the first chromosome.

### CROSS REFERENCE TO RELATED APPLICATIONS

Pursuant to 35 U.S.C. §119(e), this application claims priority to the filing date of U.S. Provisional Patent Application Ser. No. 61/482,069 filed May 3, 2011; the disclosure of which application is herein incorporated by reference.

### INTRODUCTION

Autosomal recessive disorders and predispositions to cancer can often be explained by a “two-hit” model, where the proper function of both homologous copies of a gene (one on each autosomal chromosome) is disrupted by two independent events, inherited or otherwise. Those deleterious events may be single nucleotide polymorphisms (SNPs), insertion-deletions (“in-dels”), copy number variations (CNVs), methylation, somatic variations or other epigenetic events. Unless both deleterious events are identical homozygous variants, most genotyping and sequencing methods, as they are carried out today, are generally incapable of determining whether multiple SNPs or CNVs or other variations reside within the same copy of a gene or within two distinct copies, for all but the shortest of genes. For example, none of today's array-based methods can correlate SNPs across large alleles for a given mammalian sample. Sequencing-based methods can correlate copies over short distances, comparable to the read lengths (about 1000 bases for Sanger sequencing), and some involving paired-end sequencing can correlate over longer distances, but even those are either limited to the relatively narrow distance ranges that are selected for, or involve construction of clone libraries (YACs, BACs, fosmids). Knowing whether one or two homologous copies of a gene are disrupted is important for disease diagnostics and therapeutics, and for improving our understanding of disease causation.

Of the 46 human chromosomes, all 23 pairs are diploid in normal females, as are the 22 autosomal pairs in males. Nevertheless, there is currently no viable means of determining whether two SNPs (on the same chromosome) that are a substantial genomic distance apart are correlated in phase. Autosomal recessive disorders occur when both homologous copies of a disease gene are in a mutated form. There are approximately 1000 known recessive disorders, including: cystic fibrosis, sickle-cell anemia, Parkinson's disease, Tay-Sachs disease, galactosemia, phenylketonuria, adenosine deaminase deficiency, growth hormone deficiency, Werner's syndrome (juvenile muscular dystrophy), albinism, and autism. For many of these disorders, there are multiple known variants and numerous unknown rare variants.

A new method for the phased genotyping of a diploid genome is provided.

### BRIEF DESCRIPTION OF THE FIGURES

FIG. 1 Fluorescence In-Situ Hybridization to determine copy number of various CNV loci in a daughter sample. All 4 CNVs occur as 1 copy that distinguish the maternal and paternal chromosomes. These results are consistent with microarray genotyping data. Further genotyping of the parents is needed to determine the parent-of-origin of each of these CNVs.

FIG. 2 schematically illustrates the positions of the FISH probes on chromosomes 1, 4, 6 and 8.

FIG. 3 shows oligonucleotide FISH (oFISH) results using chromosomes displayed using SmartType software.

FIG. 4 schematically illustrates the entry of metaphase cells from the left, the introduction of a protease from a channel at the top, the degradation of a cell and nuclear membranes, and the release of their cellular contents into the microchannel. Also, shown (without detail) is the selection of chromosomes based on their size as measured by their total DNA content.

FIG. 5 shows examples of parent-specific primer pairs and molecular beacons for polymorphic CNV (DGV-11397) at the coordinates: chr1:61855446-61856289 in build hg18 (NCBI build 36). Maternal (left) and paternal (right) alleles of the sample GM19240 (a.k.a. NA19240) which is heterozygous for this deletion CNV. The real-time amplification of this interval has been validated using the primers below within samples GM19238, GM19239, and GM19240, which carry two, zero, and one copies of the deletion interval, respectively. Primer 1 is a forward primer external to the deletion, and the complements of the sequences labeled primer 2 (internal) and primer 3 (external) are used as reverse primers. As the PCR is optimized for short products, only the 137 by interval from primer 1 to primer 2 is amplified in the maternal sample, suppressing the longer product. And, the paternal sample produces a product of 134 by spanning the regions flanking the deletion interval. The deletion interval is marked with < > symbols in the maternal allele sequence. From top to bottom: SEQ ID NOS: 1-4.

FIG. 6 shows the analysis of parent-of-origin of chromosomes within immiscible plugs using on-chip continuous flow PCR. Plugs with a chromosome from one parent are labeled red, and those plugs with chromosomes from the other parent are labeled green.

FIG. 7. Apparatus for the determination of parent-of-origin for chromosomal material within aqueous droplets in an immiscible medium by means of on-chip continuous flow PCR. Droplets with a chromosome from one parent are labeled red, and those droplets with chromosomes from the other parent are labeled green.

FIG. 8 illustrates an on-chip microfluidic mechanism for sorting droplets based on their fluorescence into two, or three collection chambers.

FIG. 9. Apparatus for the creation of an emulsion consisting of aqueous droplets containing whole chromosomal material, PCR primers, labeled reporters, and enzymes for PCR and possibly for fragmentation of genomic DNA. The microfluidic mechanism forms droplets that are stored as an emulsion in a collection chamber, either on-chip or off-chip.

FIG. 10. A sorting mechanism for droplets contained in an emulsion of droplets containing chromosomes and labeled by parent of origin.

FIG. 11 shows a demonstration of allele-specific realtime PCR of both genomic and chromosomal material for the primers and molecular beacons as indicated in FIG. 5. All reactions in for FIGS. 11(a), (b), (c) and (d) included all three primers as well as both internal (Cy5) and external (Cy3) reporters. FIG. XX(a) and XX(b) show signals as a function of cycle number for the trio of samples, including NA19238 (mother), NA19239 (father), and NA19240 (daughter) in the Cy5 and Cy3 channels, respectively. The input DNA quantities were 2.5 ng (corresponding to approximately whole 380 genomic equivalents) of fragmented genomic material (from Coriell Repositories) in 50 microliters. These amplification results are consistent with the previously characterized deletion interval copy numbers of two, zero and one for mother (blue), father (green) and daughter (red), respectively, and for the CNV interval DGV-11397. Water was used for the negative control (cyan). FIGS. 11 (c) and 11 (d) show real-time PCR results for the amplification of whole intact chromosomes extracted from cell line GM19240 (daughter) using only a cell lysis and without any DNA clean up or purification. In this case, the input genomic DNA quantity was estimated at 50 ng (blue), and the negative control (green) was the same buffer used for cell lysis. These results demonstrate multiplex two-color real-time PCR of whole chromosomes at concentrations equivalent to, or greater than, those of individual chromosomes within small wells or droplets.

### DEFINITIONS

The term “sample”, as used herein, relates to a material or mixture of materials, typically, although not necessarily, in liquid form, containing one or more analytes of interest.

The term “genome”, as used herein, refers to the nuclear DNA of an organism. The term “genomic DNA” as used herein refers to deoxyribonucleic acids that are obtained from the nucleus of an organism. The terms “genome” and “genomic DNA” encompass genetic material that may have undergone amplification, purification, or fragmentation. In some cases, genomic DNA encompasses nucleic acids isolated from a single cell, or a small number of cells. The “genome” in the sample that is of interest in a study may encompass the entirety of the genetic material from an organism, or it may encompass only a selected fraction thereof: for example, a genome may encompass one chromosome from an organism with a plurality of chromosomes. The terms “genome” and “genomic DNA” do not encompass cDNA (which is complementary DNA made from RNA, e.g., mRNA). However, as is well known, information about a cell's genome (e.g., about SNPs etc) can be obtained from examining cDNA from that cell.

The term “genomic region” or “genomic segment”, as used herein, denotes a contiguous length of nucleotides in a genome of an organism. A genomic region may be of a length as small as a few kb (e.g., at least 5 kb, at least 10 kb or at least 20 kb), up to an entire chromosome or more.

The terms “test”, as used herein with reference to a type of sample (e.g., a genome), refers to a sample that is under study.

The term “reference,” as used herein with reference to a type of sample, refers to a sample to which a test sample may be compared. A reference sample is generally the same species (e.g., where the species is human, or mouse, for example) as that of the test sample. The reference sample may represent an individual genome, e.g., of a cell line, or may represent either a physical pooling of the genomes of multiple individuals or a computational combination of data from a number of individuals. A “reference sample” presumes that the genotype of the reference sample is known. In some cases, the genotype of the reference sample is known from previously measured array results, or from sequencing. In other cases, the reference contains a region of known nucleotide sequence, e.g. a chromosomal region whose sequence is deposited at NCBI's Genbank database or other databases, for example.

The term “nucleotide” is intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the term “nucleotide” includes those moieties that contain hapten or fluorescent labels and may contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or functionalized as ethers, amines, or the likes. Nucleotides may include those that when incorporated into an extending strand of a nucleic acid enables continued extension (non-chain terminating nucleotides) and those that prevent subsequent extension (e.g. chain terminators).

The term “nucleic acid” and “polynucleotide” are used interchangeably herein to describe a polymer of any length, e.g., greater than about 2 bases, greater than about 10 bases, greater than about 100 bases, greater than about 500 bases, greater than 1000 bases, up to about 10,000 or more bases composed of nucleotides, e.g., deoxyribonucleotides or ribonucleotides, and may be produced enzymatically or synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions. Naturally-occurring nucleotides include guanine, cytosine, adenine, uracil and thymine (G, C, A, U and T, respectively).

The term “oligonucleotide”, as used herein, denotes a single-stranded multimer of nucleotides from about 2 to 500 nucleotides, e.g., 2 to 200 nucleotides. Oligonucleotides may be synthetic or may be made enzymatically, and, in some embodiments, are between 10 to 50 nucleotides in length. Oligonucleotides may contain ribonucleotide monomers (i.e., may be oligoribonucleotides) or deoxyribonucleotide monomers. Oligonucleotides may be 10 to 20, 11 to 30, 31 to 40, 41 to 50, 51-60, 61 to 70, 71 to 80, 80 to 100, 100 to 150 or 150 to 200, up to 500 or more nucleotides in length, for example.

The term “duplex” or “double-stranded” as used herein refers to nucleic acids formed by hybridization of two single strands of nucleic acids containing complementary sequences. In most cases, genomic DNA is double-stranded.

The term “complementary” as used herein refers to a nucleotide sequence that base-pairs by non-covalent bonds to a target nucleic acid of interest. In the canonical Watson-Crick base pairing, adenine (A) forms a base pair with thymine (T), as does guanine (G) with cytosine (C) in DNA. In RNA, thymine is replaced by uracil (U). As such, A is complementary to T and G is complementary to C. In RNA, A is complementary to U and vice versa. Typically, “complementary” refers to a nucleotide sequence that is at least partially complementary. The term “complementary” may also encompass duplexes that are fully complementary such that every nucleotide in one strand is complementary to every nucleotide in the other strand in corresponding positions. In certain cases, a nucleotide sequence may be partially complementary to a target, in which not all nucleotides are complementary to the corresponding nucleotides in the target nucleic acid.

The term “probe,” as used herein, refers to a nucleic acid that is complementary to a nucleotide sequence of interest. In certain cases, detection of a target analyte requires hybridization of a probe to a target. In certain embodiments, a probe may be immobilized on a surface of a substrate, where the substrate can have a variety of configurations, e.g., a sheet, bead, or other structure. In certain embodiments, a probe may be present on a surface of a planar support, e.g., in the form of an array. A labeled probe may be directly or indirectly connected to a detectable label.

An “array,” includes any two-dimensional and three-dimensional arrangement of addressable regions, e.g., spatially addressable regions or optically addressable regions, bearing nucleic acids, particularly oligonucleotides or synthetic mimetics thereof, and the like. In some cases, the addressable regions of the array may not be physically connected to one another, for example, a plurality of beads that are distinguishable by optical or other means may constitute an array. Where the arrays are arrays of nucleic acids, the nucleic acids may be adsorbed, physisorbed, chemisorbed, or covalently attached to the arrays at any point or points along the nucleic acid chain.

Any given substrate may carry one, two, four or more arrays disposed on a surface of the substrate. Depending upon the use, any or all of the arrays may be the same or different from one another and each may contain multiple spots or features. An array may contain one or more, including more than two, more than ten, more than one hundred, more than one thousand, more than ten thousand features, or even more than one hundred thousand features, in an area of less than 20 cm² or even less than 10 cm², e.g., less than about 5 cm², including less than about 1 cm², less than about 1 mm², e.g., 100 μm², or even smaller. For example, features may have widths (that is, diameter, for a round spot) in the range from a 5 μm to 1.0 cm. In other embodiments each feature may have a width in the range of 1.0 μm to 1.0 mm, usually 5.0 μm to 500 and more usually 10 μm to 200 Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, 20%, 50%, 95%, 99% or 100% of the total number of features). Inter-feature areas will typically (but not essentially) be present which do not carry any nucleic acids (or other biopolymer or chemical moiety of a type of which the features are composed). Such inter-feature areas typically will be present where the arrays are formed by processes involving drop deposition of reagents but may not be present when, for example, photolithographic array fabrication processes are used. It will be appreciated though, that the inter-feature areas, when present, could be of various sizes and configurations.

Arrays can be fabricated using drop deposition from pulse-jets of either precursor units (such as nucleotide or amino acid monomers) in the case of in situ fabrication, or the previously obtained nucleic acid. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, U.S. Pat. No. 6,323,043, U.S. Patent Application Publication No. 20040203138 by Caren et al., and the references cited therein. As already mentioned, these references are incorporated herein by reference. Other drop deposition methods can be used for fabrication, as previously described herein. Also, instead of drop deposition methods, photolithographic array fabrication methods may be used. Inter-feature areas need not be present particularly when the arrays are made by photolithographic methods as described in those patents.

Arrays may also be made by distributing pre-synthesized nucleic acids linked to beads, also termed microspheres, onto a solid support. In certain embodiments, unique optical signatures are incorporated into the beads, e.g. fluorescent dyes, that could be used to identify the chemical functionality on any particular bead. Since the beads are first coded with an optical signature, the array may be decoded later, such that correlation of the location of an individual site on the array with the probe at that particular site may be made after the array has been made. Such methods are described in detail in, for example, U.S. Pat. Nos. 6,355,431, 7,033,754, and 7,060,431.

An array is “addressable” when it has multiple regions of different moieties (e.g., different oligonucleotide sequences) such that a region (i.e., a “feature” or “spot” of the array) at a particular predetermined location (i.e., an “address”) on the array contains a particular sequence. Array features are typically, but need not be, separated by intervening spaces. An array is also “addressable” if the features of the array each have an optically detectable signature that identifies the moiety present at that feature. An array is also “addressable” if the features of the array each have a signature, which is detectable by non-optical means, that identifies the moiety present at that feature.

The terms “determining”, “measuring”, “evaluating”, “assessing”, “analyzing”, and “assaying” are used interchangeably herein to refer to any form of measurement, and include determining if an element is present or not. These terms include both quantitative and/or qualitative determinations. Assessing may be relative or absolute. “Assessing the presence of” includes determining the amount of something present, as well as determining whether it is present or absent.

The term “using” has its conventional meaning, and, as such, means employing, e.g., putting into service, a method or composition to attain an end. For example, if a program is used to create a file, a program is executed to make a file, the file usually being the output of the program. In another example, if a computer file is used, it is usually accessed, read, and the information stored in the file employed to attain an end. Similarly if a unique identifier, e.g., a barcode is used, the unique identifier is usually read to identify, for example, an object or file associated with the unique identifier.

The term “hybridization conditions” as used herein refers to hybridization conditions that are optimized to anneal an oligonucleotide of a sufficient length to a probe, e.g. an oligonucleotide that is not nicked and has a contiguous length of at least 20 nucleotides (e.g. at least 30, at least 40, up to at least 50 or more) complementary to a nucleotide sequence of the probe. Hybridization conditions may provide for dissociation of duplexes that anneal over a short length of region (e.g. less than 50, less than 40, less than 30, or less than 20 contiguous nucleotides) but not dissociation of duplexes formed between an un-nicked strand and its respective probe. Such conditions may differ from one experiment to the next depending on the length and the nucleotide content of the complementary region. In certain cases, the temperature for low-stringency hybridization is 5°-10° C. lower than the calculated Tₘ of the resulting duplex under the conditions used. Details on the hybridization conditions suitable for use in certain embodiments in the present disclosure may be found in US Patent Publication 20090035762, the disclosure of which is incorporated herein by reference.

As used herein, the term “data” refers to a collection of organized information, generally derived from results of experiments in lab or in silico, other data available to one of skilled in the art, or a set of premises. Data may be in the form of numbers, words, annotations, or images, as measurements or observations of a set of variables. Data can be stored in various forms of electronic media as well as obtained from auxiliary databases.

As used herein, the term “plurality” refers to at least 2, e.g., at least 5, at least 10, at least 20, at least 50, at least 100, at least 500, at least 1,000, at least 5,000 or at least 10,000 or more, up to 50,000, or 100,000 or more.

As used herein, the term “diploid” refers to a genome that exist in a cell with a copy number of two, i.e., twice the haploid number. For example, a reference assembly of the human genome includes approximately 3×10⁹ base pairs of DNA organized into distinct chromosomes. The genome of a normal somatic human cell consists of 22 pairs of autosomes (chromosomes 1 to 22) and either chromosomes X and Y (males) or a pair of X chromosomes (female) for a total of 46 chromosomes. In a typical human cell, the autosomes are diploid.

As used herein, the term “chromosomal sample” refers to a sample that contains intact chromosomes, where an “intact chromosome” is a chromosome that contains a centromere, a long arm containing a telomere and a short arm containing a telomere, and telocentric and holocentric chromosomes. As is known, chromosomal samples containing intact chromosomes can be made from an interphase or metaphase cells.

As used herein, the term “in situ hybridization” refers to hybridization of a probe to a specific nucleic acid sequence of an intact chromosome. An intact chromosome may be present inside a cell or is isolated from a cell.

As used herein, the term “in situ” in the context of hybridization refers to hybridization of a nucleic acid to a complementary nucleic acid in an intact chromosome. Suitable in situ hybridization conditions may include both hybridization conditions and optional wash conditions, which include temperature, concentration and denaturing reagents.

As used herein, the term “homologous” in the context of a pair of homologous chromosomes refers to a pair of chromosomes from an individual that are similar in length, gene position and centromere location, and that line up and synapse during meiosis. In an individual, one chromosome of a pair of homologous chromosomes comes from the mother of the individual (i.e., is “maternally-derived”), whereas the other chromosomes of the pair comes from the father (i.e., is “paternally-derived”). In the context of genes, the term “homologous” refers to a pair of genes where each gene resides within each homologous chromosome at the same position and has the same function.

As used herein, the term “isolating” refers to separating one or more chromosomes (e.g., maternally- or paternally-derived copies of chromosome 1, 2 and/or 3, etc.) from other chromosomes of a sample.

As used herein, the term “isolated”, in the context of an isolated chromosome, refers to a composition that contains one or more chromosomes (e.g., maternally- or paternally-derived copies of chromosome 1, 2 and/or 3, etc.) that have been separated from other chromosomes of a sample.

As used herein, the term “same chromosome”, as used in the context of multiple copies of the same chromosome, refers to chromosomes having the same chromosome number (e.g., chromosome 1, chromosome 2, chromosome 3, etc.). Conversely, as used herein, the term “different chromosomes”, as used in the context of a sample containing different chromosomes, refers to a sample containing chromosomes that have different chromosome numbers. For example, a sample containing at least two of the same chromosome can have two chromosome is (although other chromosomes may be present), whereas a sample containing at least two different chromosomes may have one chromosome 1 and one chromosome 2 (although other chromosomes may be present).

As used herein, the term “independently isolating”, e.g., in the context of independently isolating two chromosomes, refers to a method that results in at least two compositions, one that contains one of the chromosomes and another composition that contains the other of the chromosomes. For example, if the maternally-derived and paternally-derived copies of chromosome 1 are independently isolated from a sample, then the isolating will result in at least two distinct compositions, one containing maternally-derived copies of chromosome 1 and the other containing paternally-derived copies of chromosome 1.

As used herein, the term “independently genotyping”, e.g., in the context of independently genotyping two isolated chromosomes, refers to a method in which the isolated chromosomes are genotyped separately from one another. For example, if a sample containing maternally-derived chromosomes is independently genotyped from a sample of paternally-derived copies, then the genotyping will result in at least two distinct datasets, one for the maternally-derived chromosomes and the other for the paternally-derived chromosomes.

As used herein, the term “phasing” in the context of genotyping or sequencing (e.g. “phased-sequencing”) is the determination of the relationship between the between genotypes for multiple variants on specific parentally-derived chromosomes.

As used herein, the term “copy number variation” refers to a sequence that is present at a different copy number in a locus of one chromosome relative to the same locus in a homologous chromosome. A copy number variation can be indicated by a sequence that is present in one chromosome but not the other (i.e., is bi-allelic), or by a sequence that is present with a copy number of one in one chromosome and a copy number of more than one (e.g., 2, 3 or 4 or more) in the homologous chromosome, for example. The term “copy number variation” includes in-dels of as small as a single nucleotide.

As used herein, the term “homozygous” denotes a genetic condition in which identical alleles reside at the same loci on homologous chromosomes.

As used herein, the term, “heterozygous” denotes a genetic condition in which different alleles reside at the same loci on homologous chromosomes.

As used herein, the term “single nucleotide polymorphism”, or “SNP” for short, refers to a phenomenon in which two or more alternative alleles (i.e., different nucleotides) are present at a single nucleotide position in a genomic sequence at appreciable frequency (e.g., often 1%) in a population. In some cases, SNPs may be present at a frequency less than 1% in a population. As used herein, the term SNP may include these “rare SNPs” (present at a frequency less than 1% in a population) or even “single nucleotide variants” (SNVs) that have only been detected in one or a few samples to date.

As used herein, the term “SNP site” denotes the position of a SNP in a genomic sequence. A SNP site may be indicated by genomic coordinates. The nucleotide sequences of hundreds of thousands of SNPs from humans, other mammals (e.g., mice), and a variety of different plants (e.g., corn, rice and soybean), are known (see, e.g., Riva et al 2004, A SNP-centric database for the investigation of the human genome BMC Bioinformatics 5:33; McCarthy et al 2000 The use of single-nucleotide polymorphism maps in pharmacogenomics Nat Biotechnology 18:505-8) and are available in public databases (e.g., NCBI's online dbSNP database, and the online database of the International HapMap Project; see also Teufel et al 2006 Current bioinformatics tools in genomic biomedical research Int. J. Mol. Med. 17:967-73).

As used herein, the term “SNP allele” refers to the identity of the nucleotide at a SNP site (e.g., whether the SNP site has a G, A, T or C). A “first allele” and a “second allele” of a SNP are different alleles, i.e., they have different nucleotides at the SNP site.

As used herein, the term “allele-specific copy number” indicates the number of copies of a particular SNP allele in a cell of a sample.

The term “chromosomal aberration” refers to a difference between the chromosomes of a test sample and a reference sample. Examples of chromosome aberrations include chromosomal rearrangements, e.g., inversions, translocations, duplications, deletions and insertions, etc.

The term “data” refers to both raw data and processed data. Raw data may be processed, e.g., normalized, smoothed, filtered, etc., prior to use in the subject method using any suitable method (see, e.g., Quackenbush, Nat. Gen. 2002 Supp. 32, van Houte et al BMC Genomics. 2009; 10:401 and Staaf et al BMC Genomics. 2007 8:382, Staaf et al BMC Bioinformatics. 2008 9:409, Rigaill et al Bioinformatics. 2008 24:768-74, Curry et al Normalization of Array CGH Data In Methods in Microarray Pages 233-244 Normalization CRC Press 2008; incorporated by reference for all data processing steps, among many others).

As used herein, the term “genotyping” is intended to be a separate activity relative to the step in which the parent of origin of a chromosome is determined.

The term “SNP assay” refers to an assay in which the SNPs of a test sample are analyzed in order to determined which SNP alleles are present in the test sample. Such an assay may be done by a wide variety of methods, including those of US20090035762, Mei et al (Genome Res. 2000 10: 1126-37) or Gunderson et al (Nat. Genet. 2005 37:549-54), for example. In one embodiment, the assay may be done by sequencing a sample. In one embodiment, the assays involve comparing the level of hybridization of a test sample to a SNP-discriminating oligonucleotide relative to the level of hybridization of a reference sample to the same oligonucleotide. The ratio of hybridization indicates the relative numbers of copies of one of the SNP alleles present in the sample and the reference.

The terms “CGH assay” and “comparative genomic hybridization assay” refers to an assay in which the relative copy number of the same locus in two samples (e.g., a test sample and a reference sample) is determined. The general principles of a CGH assay are described in Barrett et al (Proc Natl Acad Sci 2004 101:17765-70) and Hostetter et al (Nucleic Acids Res. 2010 38: e9), for example. Such assays involve comparing the level of hybridization of a test sample to an oligonucleotide relative to the level of hybridization of a reference sample to the same oligonucleotide. The ratio of hybridization levels indicates the relative copy numbers of a sequence in the sample.

The term “biallelic CNV” refers to a region of the genome known to be copy number variant and polymorphic in a population and to exist primarily in two common allelic states. Thousands of examples of such biallelic CNVs have already been reported in various publications, e.g., Campbell et al (AJHG 2011, 88:317-332), Li et al (Nat. Biotechnol. 2010 28: 57-63) and Kidd et al (Nature 2008 453: 56-64).

### DESCRIPTION OF EXEMPLARY EMBODIMENTS

Before the present invention is described in greater detail, it is to be understood that this invention is not limited to particular embodiments described, and as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

A method of sample analysis is provided. In certain embodiments the method comprises: a) obtaining from a diploid individual a chromosomal sample that comprises maternally-derived chromosomes and homologous paternally-derived chromosomes; b) determining the parent of origin of a first chromosome of the sample by detecting a parent-specific copy number variation relative to a second chromosome that is homologous to the first chromosome; c) isolating the first chromosome after its parent of origin is determined; and d) genotyping the isolated chromosome of step c).

In certain cases, the determining may comprise determining the parent of origin of a plurality of chromosomes in the sample, the isolating comprises pooling at least two chromosomes (e.g., up to 1,000 or more chromosomes) of the same parental origin; the isolating comprises genotyping the at least two chromosomes of the same parental origin. The pooled chromosomes may be the same chromosome (i.e., they may have the same chromosome number), or they may be different chromosomes (i.e., they may have the different chromosome numbers).

In particular cases, the copy number variation is a nucleotide sequence that is present in one of the maternally-derived or homologous paternally-derived chromosomes and not present in the other of the chromosomes.

In certain embodiments, the genotyping step (which is performed as a distinct step from the parent of origin analysis) comprises sequencing at least part of the first chromosome. The genotyping can be done by array analysis or by PCR, for example.

In certain embodiments, the parent of origin analysis may be done using an in situ hybridization method. In these embodiments, the method may comprise hybridizing to the chromosomal sample, in situ, a labeled nucleic acid probe that differentially hybridizes to copy number variation that distinguishes a maternally-derived chromosome and a homologous paternally-derived chromosome; isolating the maternally-derived chromosome or the paternally-derived chromosome from other chromosomes in the sample on the basis of the labeling to produce an isolated maternally-derived chromosome or an isolated paternally-derived chromosome; and genotyping the isolated maternally-derived chromosome or the isolated paternally-derived chromosome.

In certain embodiments, the parent of origin analysis may be done using PCR. In these embodiments, the method may comprise: isolating the individual chromosomes; determining the parent of origin of a chromosome by polymerase chain reaction (PCR); and genotyping the chromosome. In some cases, the parent of origin analysis may be done by depositing individual chromosomes into separate wells, and performing PCR analysis. In these embodiments, the method may comprise depositing individual chromosomes of the sample into separate wells; determining the parent of origin of a chromosome by polymerase chain reaction; and genotyping the chromosome. In other embodiments, the method may comprise separating the chromosomes into discrete plugs in the flow stream of a microfluidics device; determining the parent of origin of the first chromosome of the sample by PCR in the plug, wherein the parent of origin of the first chromosome is indicated by fluorescence; collecting a plug containing a chromosome on the basis of its fluorescence; and genotyping the collected first chromosome.

In some case, the method may comprise pooling at least ten chromosomes of the same parental origin, wherein the at least ten chromosomes are the same chromosome and the genotyping comprises subjecting the pooled sample to at least two different genotyping methods. In some embodiments, the genotyping may comprise determining the relative copy numbers of sequences or determining the status of SNPs in the first chromosome. In other embodiments, the genotyping may comprise determining the methylation status or histone modification status of the first chromosome.

Further details of the method, including methods for identifying parent-specific markers, methods for identifying and isolating parent-specific chromosomes, and methods for genotyping isolated parent-specific chromosomes are set forth below.

The human genome is diploid, and the genome of a typical individual has hundreds of thousands of distinct differences across the 22 pairs of homologous autosomal chromosomes. These differences come in the form of variants, such as SNPs, indels and CNVs. Although conventional sequencing methods can generally determine which of those SNPs and CNVs are heterozygous (i.e. across the pair of homologous chromosomes upon which they reside), they can generally not determine which heterozygous variants are on the same homologous chromosome as other heterozygous variants on that chromosome pair. Or, stated another way, many conventional methods cannot determine from which parent the variant originates. Differences between these homologous chromosomes can be used to differentiate one chromosome from the other. Some embodiments of the method described herein solve this problem.

In some methods, multiple copies of the same homologous chromosome (or sets of chromosomes) can be pooled before genotyping, e.g., before amplification and sequencing, giving this approach several advantages over other prior methods that either sequence individual chromosomes or sequence clone pools without a priori knowledge of parental origin before genotyping. For example, in certain embodiments, high-throughput sequencing can be applied without the requirement for barcoding each independent sample (or its amplicons), with more even representation of sequences (or amplified product) across each chromosome. The more copies of each chromosome that can be isolated the less amplification gain that is needed prior to sequencing, and the more uniform the representation. Certain other methods that amplify individual chromosomes are fraught with non-uniformity, with some genomic regions amplified greatly (inefficiently using the sequencing resources) and either under-represented or missing all-together. In one embodiment, the method involves collecting of multiple copies of identified chromosomes in solution using a microfluidic system. In many embodiments the principle means of detection is amplification of known parent-specific variants (such as a CNVs, novel sequences, indels, or, in some embodiments, SNPs), and labeling the product of that amplification. The amplification products indicate the origin of the whole chromosome, which is subsequently isolated and pooled with previously isolated chromosomes with the same parent of origin.

### Identification of Parent-Specific Markers

In performing the subject method, a chromosome is analyzed to determine whether it is maternally derived or paternally derived, prior to genotyping. This parent of origin analysis step of the method may be done, e.g., using a probe that differentially hybridizes to the maternally-derived chromosome of a pair of homologous chromosomes relative to the homologous paternally-derived chromosome of the pair. The site to which the probe binds in a chromosome pair may be thought of as a “parent-specific marker”. Binding of the probe to such a marker (where the marker may be present in an intact chromosome or in a product amplified from a chromosome) identifies which parent a chromosome is derived from.

Parent-specific markers can be identified by a variety of different methods. In certain embodiments, parent-specific markers can be identified by analyzing the genome of the parents of the individual from which the chromosomal sample is obtained (e.g., by CGH) to identify, e.g., a sequence that differs in copy number (i.e., a CNV) between the parents, and that is homozygous in both parents. Assuming that the chromosomes are inherited in a Mendelian way, the individual will be heterozygous for the CNV and the two different homologous chromosomes can be distinguished by the presence or absence of the sequence within the CNV. For example, if one parent is homozygous for the existence of the sequence and the other for its absence, then the origin of each homologous chromosome in the individual can be unambiguously assigned by determination of its presence or absence. In a similar way, parent-specific markers can be identified by analyzing the genome of one parent of the individual from which the chromosomal sample is obtained as well as the individual. Further, in certain cases, parent-specific markers can be identified a priori, i.e., prior to the parent of origin analysis, or ad hoc, i.e., as the parent of origin analysis is being done.

In one embodiment, the parent-specific marker may be identified a priori, i.e., before the parent of origin analysis is done. Specifically, this embodiment involves identifying in the individual at least one heterozygous parent-specific allele for each chromosome of interest before the parent of origin analysis is done. In one embodiment, more than one variant can be identified for each chromosome. Such up-front genotyping of the sample (or its parents) can be performed by, e.g., performing CGH analysis on the sample before informative variant identification. If both parents are homozygous for two different alleles of the same variant, then the progeny chromosome would necessarily (by Mendelian inheritance) be heterozygous for that variant. Other means of a priori genotyping include, for example: PCR, multiplex PCR, sequencing, and target enriched sequencing, etc.

In another embodiment, the parent-specific marker may be identified ad hoc, i.e., during the parent of origin analysis step. This embodiment may be employed when the sample genotypes are not pre-screened. In some cases, this embodiment entails identifying at least one differential (heterozygous) genetic marker for the chromosome of interest during the parent of origin analysis. In this approach, markers for multiple variants are distinguishably labeled, for using distinguishable fluorophores or quantum dots, without prior knowledge as to which ones are heterozygous and the phasing relationships between variants existing on parentally-derived chromosomes. In this embodiment, the informative variants are only identified during the analysis step. Once the markers that are informative for that specific chromosome have been identified, only probes for the markers are used in subsequent chromosome isolation of the same sample. In this embodiment, the marker alleles used and their labeling may need to be altered between runs on the same sample. In this embodiment it is not necessary to know the parent of origin prior to initiating the analysis. Rather, one should simply determine that one homologous chromosome is distinct from the other.

In certain cases, for alleles that are biallelic and follow Mendelian inheritance, only two of the three samples (mother, father and child) may be genotyped to properly identify parent specific markers. If only one parent and the child are genotyped, and that parent is homozygous and the child is heterozygous for a given variant, the origin of the allele can be accurately determined. Specifically, if the genotyped parent is homozygous with two copies present, the child should have inherited the one copy of the allele from that parent. Conversely, if the genotyped parent is homozygous with no copies present, then the child inherited its one copy from the other parent.

If the child is not genotyped, then the variants that can be definitively determined to be heterozygous for a single copy in the child are those for which one parent is homozygous with two copies and the other parent is homozygous with zero copies of the variant interval. Of course, those for which one parent is homozygous for a loss (zero copies) may still be informative (50% of the time) if the other parent is heterozygous (with a single copy). Thus, probes for these intervals can be useful and worthy of parent-specific labeling as well, in the absence of the child's genotype.

Small “in-dels” (i.e., insertions & deletions) may exist in one copy of a chromosome copy, and be absent from the other copy. The identification of a heterozygous simple-deletion variant that exists only on one, say the maternally-derived, chromosome but not on the other (paternally-derived) copy indicates that variant can be used to uniquely identify the parental origin of the chromosome. In one embodiment, the present allele may be identified by, for example, oligo-FISH (e.g., Yamada et al. Cytogenet Genome Res. 2011 132: 248-54; see also 20110039735, 20100221708, 20100068701 and 20100055681). By combining this approach with conventional chromosome-specific markers, such as centromeric BAC or oligo-FISH markers, G-band staining, chromosome barcoding, all chromosomes can be uniquely identified.

In the simplest form of this method any individual heterozygous (single copy) CNV can be used to identify a parent-specific chromosome that contains the variant sequence, and the second copy of that homologous chromosome (absent the variant sequence) can be determined by a second chromosome specific probe, e.g. a centromeric probe. In this case, only the unknown sample need be pre-screened by conventional (such as array based) copy-number genotyping.

However, for redundancy, in some cases it may be advantageous to identify more than a single heterozygous variant for each chromosome pair. In some cases, each parent-specific chromosome may have at least one positive (present) allele and at least one negative (deleted) allele. Such redundancy should significantly improve the overall accuracy and efficiency of the chromosomal identification. For example, one positive allele could be used to identify a chromosome of maternal origin, and a second positive allele of opposite phase and differently labeled can identify a chromosome of paternal origin. In this example, it is possible to positively identify each of a pair of homologous chromosomes by parent of origin.

Given a set of potentially informative candidate marker alleles, an unknown sample can be genotyped for heterozygous markers. Any one of these heterozygous markers can be used to differentiate a chromosome originating in one parent from its homolog originating in the other parent. If the sample and either parent, or alternatively, both parents are genotyped (for this candidate set), then sets of heterozygous chromosomally-phased alleles can be unambiguously determined. By doing this multi-sample genotypic screening, multi-marker-allele sets can be identified for each chromosome for use in subsequent redundant (multi-color) chromosome isolation. If marker alleles are not phased for a given chromosome, then either only a single marker-allele may be used for that chromosome, or each variant allele may be labeled with a distinct color (for that same chromosome). On the other hand, if the markers are properly phased, then the same color dyes or tags can be shared by different marker alleles.

Another means of identifying the phasing for a candidate set haploid-specific markers for an unknown sample involves performing multi-color metaphase FISH where each marker variant (on a given chromosome) is labeled in a distinct color. Visual inspection of the two copies of the chromosome of interest reveals the chromosome phasing of the markers, as long as the marker alleles are of sufficient genomic distance apart that they are spatially resolvable by fluorescence microscopy. All markers on the same chromosome copy are thus “in-phase”. This method does not determine the parental origin of each chromosome, nor does it enable parental phasing across different chromosomes. However, it does make possible the selection of phased-markers for each chromosome to which it is applied, and thus enables haplotype-specific genotyping. Furthermore, when combined with G-banding, chromosome barcoding, or distinctly-labeled centromeric probes, it provides a multiplex approach to determine phasing across multiple chromosomes simultaneously.

The individual from which the chromosomal sample is obtained may be male, female, and may be of any species that has a diploid genome. An individual may be mammalian, e.g., human, mouse, rat, etc., although an individual from other species (e.g., yeast, insects, plants, birds, C. elegans, etc., may be employed). Furthermore, the sample may in certain cases contain a complete complement of chromosomes from an individual, including all autosomes and sex chromosomes, prior to labeling. There is no requirement that parent-specific markers be identified for all chromosomes if all chromosomes are not being labeled and isolated in the method. Nevertheless, the methods described above can be readily generalized to uniquely identify any subset of the 46 allele-specific chromosomes by identifying both the parental-specificity and the chromosomes.

Any of the following polymorphic markers can be used as a parent-specific marker. A CNV (or in-del) region for which one or more FISH probes hybridize within the region. For example, oligo FISH is capable of reliably detecting genomic regions as small as 5,000 basepairs (bp) using conventional microscopy used for karyotyping. So, one embodiment utilizes hemizygous alleles of 5 kb or greater in length. In some cases, the method may use a number of strongly-associated small in-dels within a haplotype block for which one or more FISH probes more strongly hybridize to one allele than to the other and where the combination provides a sufficient signal to be detected. In this case the in-dels may have common allele specificity, i.e. all present or all absent for the same allele within a haplotype block across a population of samples. In certain cases, the method may use a number of strongly-associated SNPs within a haplotype block for which one or more short oligonucleotide FISH probes hybridize within each region. The combination should provide a detectable signal.

There are more than 20,000 CNVs and many tens of thousands of in-dels in the Database of Genomic Variants (DGV, recently superseded by dbVar which contains about 500,000 variants). At least 70% of these CNVs are 5 kbp or longer, and these alleles are of sufficient size for reliable detection by PCR or oligo-FISH hybridization methods on metaphase chromosomes. The frequencies of a variant (across a population of samples) determines the informativity of that variant in an unknown sample. The higher the minor allele frequency, the higher the likelihood it will be heterozygous in an unknown sample. For example, a biallelic variant with a 50% allele frequency in a population will have a 50% probability of being heterozygous for a variant drawn from that population.

With as few as five (non-associated) variants on a given chromosome with a frequency of 20% or greater, the probability of them all being uninformative (i.e. both copies having the same allele for all variants) is less than 1%. And, for 10 such variants, the probability of all being uninformative is less than on the order of 0.001%. Also, about 20% of CNVs are bi-allelic (simple) deletion type intervals for which copy numbers of 0, 1 or 2 can be readily determined with high confidence by CGH analysis. So, with hundreds or thousands of CNVs to select from, it should be relatively straightforward to find a sufficient number of CNVs per chromosome of sufficient size, copy numbers, and frequencies that reliably identify differentiating alleles. Thus, several hundred CNVs should be more than sufficient to prescreen a sample for viable marker alleles.

### Parent of Origin Analysis

As will be discussed in greater detail below, the method may be done by any suitable analytical method, e.g., using in situ hybridization or using a PCR-based method. Exemplary methods for performing the parent of origin analysis are set forth below.

### In Situ Hybridization-Based Methods

After parent specific markers are identified, e.g., using the method set forth above, a labeled probe is hybridized to a chromosomal sample in situ, where the probe differentially hybridizes to a maternally-derived chromosome and a homologous paternally-derived chromosome, thereby providing a labeled sample in which the maternally-derived chromosome and the homologous paternally-derived chromosome are distinguishably labeled. After the chromosomes are labeled, the method involves isolating one or both of the maternally-derived chromosome and the paternally-derived chromosome from other chromosomes in the labeled sample on the basis of the labeling to produce an isolated maternally-derived chromosome and/or an isolated paternally-derived chromosome.

In particular embodiments, the labeled nucleic acid probe may hybridize to a copy number variation, e.g., to a sequence that is present in one of the maternally-derived or homologous paternally-derived chromosomes and not present in the other. In particular embodiments, the nucleic acid probe may be labeled with a fluorescent label, although other labels (e.g. quantum dots, magnetic labels, etc.) may also be employed. After the chromosomes are labeled, the parent specific chromosomes may be isolated by any of a variety of different methods, e.g., by flow cytometry, magnetic cytometry, laser microdissection or by manual manipulation. These methods are known to those skilled in the art (see, e.g., Ferguson-Smith et al, Eur. J. Hum. Genet. 1997 5: 253-65; Cygi et al, Nucl. Acids Res. 2002 30: 2790-2799; Trask et al, Science 1985 230: 1401-1403; Trask et al, Hum. Genet., 1988 78: 251-259; Arkesteijn et al, Cytometry 1995 19: 353-360; Kwak et al, Cytometry, 1994 17: 26-32; Dudin et al. Hum Genet. 1988 80:111-116).

In one embodiment, once a set of phased marker alleles has been identified for each chromosome of interest, the sample cells may be permeabilized or lysed, hybridized with allele-specific markers, and washed to remove unbound labeled probe. At this point, the cells can be either inspected by metaphase FISH or they can be lysed to release intact chromosomes in liquid phase to a flow cytometer for subsequent selection.

Once at least one of the two parent-specific chromosomes has been distinctly labeled, either or both of the chromosomes can be isolated by any convenient chromosome isolation method, such as, for example, by flow cytometry (Yu et al Nature 1981 293: 154-155 and other references cited above), or magnetic cytometry (Dudin et al. Hum Genet. 1988 80:111-116), laser microdisection, or by manual manipulation. For all of these isolation methods, the sample cells of interest may be either induced into metaphase or the selection targets a subpopulation of cells that are in metaphase. The cell membranes are chemically permeabilized or lysed in order to allow the hybridization of labeled nucleic acid probes (e.g., oligonucleotide or BAC probes) that are specific for the heterozygous marker allele(s) for the chromosome of interest. These probes are tagged with a fluorescent label, a quantum dot, detectable particle, ferrous or magnetic bead, or ligand that enables their subsequent isolation.

Once labeled, each of the two chromosomes can be detected manually by visual inspection with a microscope, or by an automated device, such as a flow cytometer, or an automated vision system, or by a magnetic device (in the case of labeling with a ferrous or magnetic particle). Isolation of the chromosomes can be accomplished by flow cytometry, laser capture microdisection, or even by manual micromanipulation and nanopipetting, such as is routinely used in ICSI (intracytoplasmic sperm injection) and in vitro fertilization. The latter approach may only be practical if relatively few copies are needed for the downstream genetic analysis, such as those employing single-molecule sequencing, or PCR amplification. Or, if the chromosomes are labeled by magnetic particle or ferrous beads, they can be isolated by application of a magnetic field or field gradient. Several of these isolation methods require the lysing of the cell membrane and flowing or manipulation of intact metaphase chromosomes during collection. These methods are known to those skilled in the art.

In one embodiment, a surface of small removable pads are used upon which metaphase chromosomes may be adhered similarly to a metaphase chromosome spread (as in karyotyping) but on a MEMS surface rather than a slide. Chromosomes that are bound to the pads can be inspected by a light microscope and those that are within the confines of a single pad and that have the appropriate tags can be isolated by launching the pads from the surface. The energy for launching the pads can be applied externally by means of a laser pulse and absorbance within a volatile material in a compartment under the pad.

Depending on how the labeled sample is to be analyzed, one or more parent-specific chromosomes may be labeled. In one embodiment, only one chromosome of the sample is labeled in a parent-specific manner (e.g., either the maternal copy or the paternal copy of chromosome 1, 2 or 3, etc.). In another embodiment, both the maternal and paternal chromosomes of a pair are labeled so that they can be distinguished and independently isolated. In other embodiments, more than one chromosome may be labeled in a parent-specific manner such that a plurality of different maternally-derived chromosomes can be distinguished from their paternally-derived counterparts. In one embodiment, the complete complement of chromosomes of an individual may be labeled so that all of the chromosomes that are derived from the mother of the individual can be distinguished from the chromosomes that are derived from the father of the individual.

Specifically, many applications, such as genome-wide association studies or diagnostics for Mendelian recessive disorders or diagnostics for multigenic disorders, may analyze an entire genome, but with parental-origin specificity. In these embodiments, when haplotyping all 46 human chromosomes, it is not necessary to isolate them into 46 distinct pools. Rather, once the differentiating heterozygous variants have been identified, each haploid chromosome can be arbitrarily pooled into one of two pools, each comprising 22 autosomes and one sex chromosome, not necessarily from the same parent. Alternatively, a subset of haploid chromosomes may be isolated into a first pool and the remainder into a second pool. Each pool would then represent an artificially-defined haploid genome. If at least one parent and the sample have been genotyped, then variants can be selected and labeled in at least one color (preferably into two or more colors) according to their parental-derivation, and can thus be assigned to pools consistent with their parental origins and hence genotypes. For example, in a two-color allele-specific assay, red can be assigned to alleles inherited from the paternal chromosome and green to the maternal. In this way, these two haploid genomes could conveniently be identified as “maternal” and “paternal”. If only certain subsets of chromosomes are of interest, then only variants for those chromosomes are probed, and the remainder are placed into a third “waste” pool.

This step of the method may employ a solid phase substrate such as slides or beads, for example. In these embodiments, a hypotonic solution may be added to metaphase cells. The cells swell, and they break open and release their chromosomes onto the substrate, e.g., a conventional glass or polymer coated slide or micron scale glass beads. Such beads can be selected for having the appropriate signals and isolated using flow sorting techniques, known to those skilled in the art.

Specifically, this step of the method may be done using a variety of different methods. In certain embodiments, FISH detection may be used. This method may involve: a) dropping hypotonically swollen cells in metaphase onto a substrate (e.g., a slide, membrane, etc.); b) hybridizing metaphase chromosomes with fluorescent probes both chromosome and allele specific; c) identifying by oligo-FISH one or both parental heterozygous alleles; d) isolating chromosomes using any of the following: i. isolation of targeted chromosomes by laser microdisection of chromosomes, ii. micromanipulation and chromosomes collection by nanopipetting. In other embodiments, detection and isolation may be done by flow cytometry in solution phase, e.g., by: a) preparing cells by enriching the metaphase population (optional for samples with a practical population already in metaphase); b) permeabilizing or lysing the cells; c) permeabilizing or lysing nuclear membrane; d) optionally cross-linking DNA within chromosomes by chemical agents; e) preparing chromosomes for hybridization (denature proteins, fragment DNA); f) hybridize with allele-specific FISH markers and g) isolate chromosomes in solution phase using flow. In some embodiments, chromosomes bound individually to glass beads detected, isolated and selected by flow cytometry, e.g., by: a) mixing isotonically expanded metaphase cells with micron-scale glass or polymer beads; b) spinning down or agitating the mixture to rupture cells and fix the metaphase chromosomes to the beads; c) hybridizing the metaphase chromosomes with fluorescent probes both chromosome and allele specific; d) identifying beads by oligo-FISH targeting one or both parental heterozygous alleles; and e) isolating beads in solution phase using flow. Such methods may be readily adapted from protocols that are known in the art.

One of the challenges in isolating chromosomes in solution phase is maintaining the integrity of the condensed chromosomes while simultaneously making the DNA accessible for DNA-DNA duplex formation during hybridization with the oligonucleotide FISH probes. For this purpose, the denaturing of protein complexes (such as histones) of the chromatin structure and/or chemical cross-linking of chromosomal DNA may be beneficial in some cases. The degradation of proteins can be accomplished enzymatically by means of proteases or by chemical agents. The cros slinking of DNA may also be accomplished by chemical agents (e.g., alkylating agents: 1,3-bis(2-chloroethyl)-1-nitrosourea (BCNU, Carmustine), which forms interstrand cross-links with DNA at N⁷ position of guanine; nitrous acid, which forms DNA cros slinks amino group of exocylclic N² of Gaunine at CG dimers; aldehydes, such as acrolein and crotonaldehyde, which form DNA interstrand crosslinks in DNA and guanine adducts of DNA can also react with protein. Schiff base formation between proteins and aldehydes cause DNA-protein inter-strand linkage; formaldehyde (HCHO), which induces protein-DNA and protein-protein crosslinks that may be reversed by incubation at 70° C.; dehydroretronecine diacetate (DHRA); 2,3-bis(acetoxymethyl)-1-methylpyrrole (BAMP); dehydromonocrotaline and dehydroretrorsine). The frequency of the cross-linked bonds needs only be sufficient to hold the chromosomes more-or-less intact, but should be low enough so as not to interfere with the hybridization of FISH probes or to preclude the downstream sequencing (or genotyping) of the DNA. In some embodiments, such as formaldehyde, the crosslinking reaction is reversible, either by cleaving the cross-linker or removing it from one or both of the strands that it joins.

In some embodiments, the crosslinking agents cross-link the DNA to proteins or protein complexes. Chemical agents such as formaldehyde have proven useful to elucidate the internal 3D structure of chromosomes and of nuclei, such as in methods 3C and Hi-C. In these embodiments the DNA is covalently cross-linked to proteins that are bound by affinity under biological conditions to DNA. Some of the proteins are consequently bound to other proteins. This approach maintains the structural integrity of the chromatin.

### PCR-Based Methods and Device for Performing the Same

In other embodiments, the parent of origin analysis may be done by PCR In one embodiment that uses PCR, the individual chromosomes of the chromosomal sample may be separated into separate reaction chambers (e.g., wells of a plate, or plugs of a microfluidic device) and the parent of origin of a chromosome is determined by PCR. In certain embodiments, the PCR may employ parent-specific primers (i.e., a pair of primers in which one of the primers hybridizes to a parent-specific sequence). In these embodiments, the parent of origin of a chromosome can be determined by the presence or absence of a product. In other embodiment, the PCR may amplify a parent specific sequence. In these embodiments, the parent of origin of a chromosome can be determined by the presence or absence of a sequence in the product. In either of these assays, molecular beacon probes, Taqman probes, FRET probes and scorpion probes can be employed to detect the presence of a product using fluorescence. Such methods may be multiplexed as desired, and in certain cases, chromosomes from one parent may be indicated by a first fluorophore (e.g., Cy3), where chromosomes from the other parent may be indicated by a fluorophore that is distinguishable from the first fluorophore (e.g., Cy5).

The microfluidic device mentioned above in certain cases may comprise a fluid flow path comprising an aqueous solution of metaphase chromosomes; a reservoir of reagents connected to the fluid flow path, comprising PCR reagents for detecting the parent of origin of at least some of the chromosomes by PCR, and chromatin digestion reagents; and a reservoir of an immiscible fluid connected to the fluid flow path via a valve, wherein the valve is controlled to produce plugs, separated from one another by the immiscible fluid, each comprising the DNA of a single metaphase chromosome and the PCR reagents. In certain embodiments, this device may further comprise a thermocycling device to perform “in plug” PCR. The device may also comprise a plug collection chamber for collecting the plugs.

In certain embodiments, the microfluidic device may further comprise a gating mechanism for separating the plugs based on fluorescence. In particular embodiments, the gating mechanism may comprise passing the plugs through a nozzle to produce a stream of droplets, and deflecting the droplets by applying a charge.

In certain embodiments, this method may involve isolating individual metaphase chromosomes, identifying the parent of origin of a chromosome, sorting the chromosomes by their parent of origin, pooling multiple copies of each chromosome, optionally amplifying genomic material, and genotyping (e.g., sequencing) the pooled material.

In certain embodiments, the method may comprise lysing cells to release whole metaphase chromosomes into solution; optionally purifying chromosomes to minimize or eliminate cellular debris and optionally size selecting chromosomes to enrich chromosomes of interest; and independently isolating chromosomes as an aqueous droplet confined by an immiscible fluid. This droplet may also include reagents, e.g., polymerase, primers and reporters, necessary for amplification and labeling within each capsule or, if the droplet is porous (e.g., an alginate capsule), the reagents can be infused after encapsulation. This method may further comprise amplifying one or more parent-specific variants, e.g. using PCR, identifying chromosomes using a fluorescent reporter (e.g. a molecular beacon probe), isolating only droplets that contain the desired signal, collecting multiple chromosomes in a pool, and then genotyping the pooled chromosomes, e.g., by sequencing. After the chromosomes are pooled, the method may comprise optionally amplifying the pools of DNA using, e.g., whole-genomic amplification methods and removing specific targeted PCR-amplification products.

In this method, the allelic chromosomes are identified before pooling, and pooling is done before genotyping. In certain embodiments, a chromosomal pool can be split arbitrarily enabling two or more distinct types of measurements to be performed on the same genomic material enabling allelic phasing that goes beyond the sequence information itself. For example, both the sequence and the methylation status (using methods such as chromatin-IP-sequencing; “CHIP-Seq”) of that sequence can be determined allele-specifically. This method makes possible combined phased Genome-Wide Association Studies (GWAS) with allele-specific epigenomics (imprinting). For example, this information can be used in large-scale studies to study genetic susceptibility to environmental causes of epigenetic modifications. Further, if sufficient quantities of the chromatin structure remain intact, for example by cross-linking the proteins and the genomic DNA, then more epigenetic histone modifications could be phased as well. This could potentially lead to new discoveries by untangling environmental effects and heritable risk factors for complex diseases.

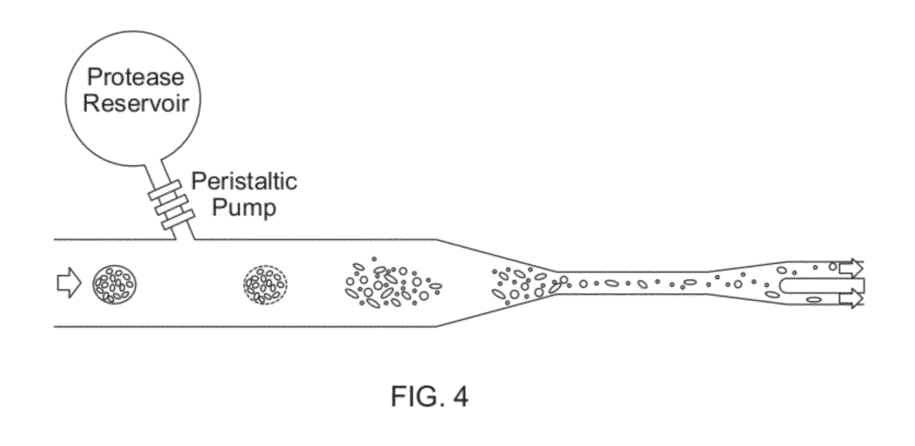

As noted above, a microfluidic device for performing certain steps of the method is provided. In certain embodiments, a population of cells that are at the metaphase of the cell cycle may be applied to the system, and, as the cells flow through the system the cells and the nuclear membranes of the cells may be chemically lysed using reagents, such as proteases that digest the cell membrane, resulting in the spilling of cell contents into the liquid medium. Those cells that are in metaphase will produce whole separable chromosomes into the liquid medium along with other cellular components. Again, by means of a stain or dye, these chromosomes may be detected, for example by imaging, while travelling down the microchannel. Optionally, the total DNA in each chromosome may be detected and the chromosomes may be size selected, keeping those within a size range of interest, as depicted in FIG. 4. This again improves the enrichment of metaphase chromosomes of interest. One mechanism for diverting the chromosomes within a desired size range will be described in the next section as it is applied to chromosome selection.

Enrichment of chromosomes of interest can be achieved via chromosome size selection. In this case the total quantity of DNA in the chromosomes can be detected using a fluorescent stain, such as DAPI or ethidium bromide, and detected by a detector or imaged by a vision system. Objects that are identified as approximately the size of the chromosomes of interest can be diverted in the selection channel using an apparatus with either two valves or the diverter channels described previously. This is an optional step, but it is useful as it eliminates much of the cellular debris that can clog the fluidic system, and it should increase the overall efficiency of the system.

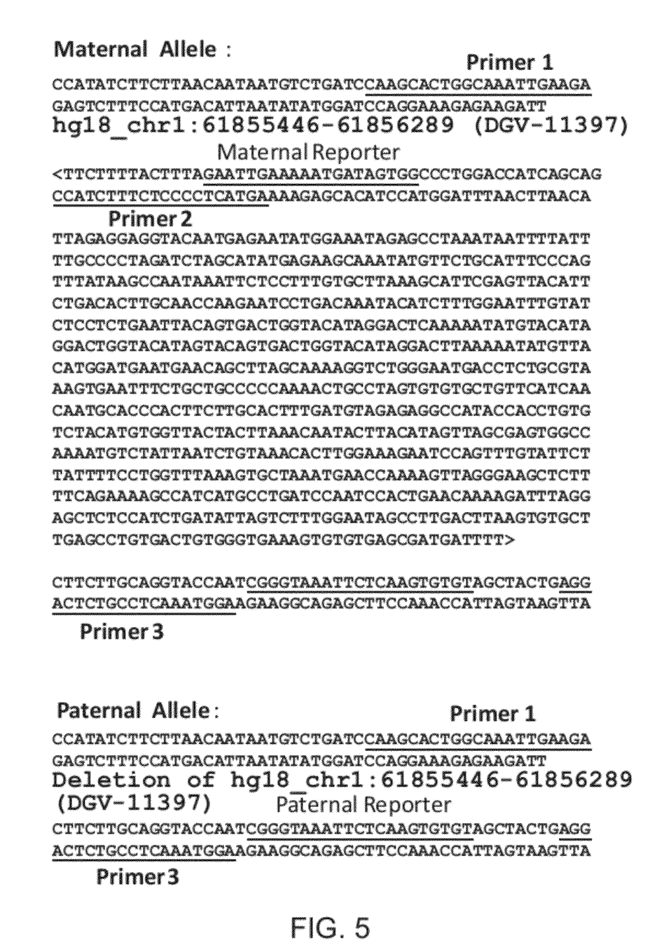

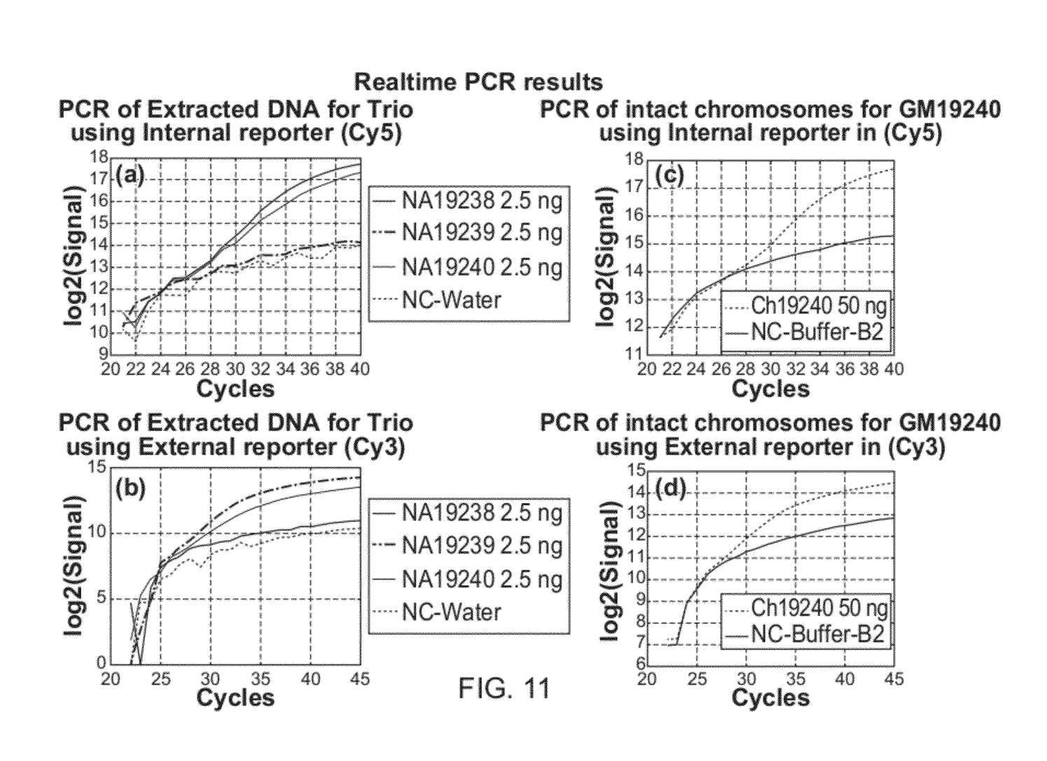

The identification of the parental origin of a homologous chromosome relies on knowing the genotype of the individual under test for at least one genomic variant per chromosome (of the 23 pair of chromosomes) to be isolated. To be most informative, these variants should be biallelic (only two known allelic states) and heterozygous in the individual under test. If a single variant is to be used for chromosomal identification, it is not necessary to know the parental genotype to distinguish the two homologous chromosomes, but only to genotype the sample under test to identify its heterozygous variants. Although biallelic copy number variants may be ideal for this application, single nucleotide variants (SNPs) could be used. Once the informative variants for each chromosome have been identified, PCR primers and reporters for those variants are manufactured, or retrieved from an inventory of primers for well-characterized polymorphisms. To positively identify both parents, two pairs of primers (or one triplet) may be needed for each variant. One pair of primers targets the allele for one parent of origin, while the other pair targets the second allele for the other parent. Three primers may be used, as two pairs may share one common primer in the same direction, as depicted in FIG. 5. For the case of a CNV or indel, a common primer could be used for both pairs. In this case, the common primer would reside outside the CNV and one of the other two primers would reside inside the CNV near the same boundary as the common primer, and the other primer would lie just outside the CNV at the opposite end of the CNV interval. For optimal multiplexing, the two amplified products of the two alleles should be short and as similar in length as possible. This will make the assay robust as long as the PCR conditions are optimized for targets of this common length (see, e.g., FIG. 11 for a reduction to practice). Additionally a reporter probe, such as a molecular beacon, can be used to identify the proper allele within the microfluidic system. And, by the use of reporter probes with two or more distinguishable labels the maternal and paternal alleles can be independently and positively idententified.

It should also be possible to achieve similar results with shorter variants, even with SNPs, although in the case of SNPs, the primers may be designed to span the variant site, and the primers and assay conditions should be optimized for each variant to ensure robust differential amplification and proper target identification.

If isolation of multiple allele-specific chromosomes (e.g. all maternal autosomes) is desired, then multiple informative variants must be identified and their associated primers fabricated and validated. This embodiment may be optimally done using a set of hundreds pairs of optimized primers for previously validated polymorphic variants, ideally with high minor allele frequencies. When both homologous copies of the same chromosome are desired, then the use of only two distinct dye molecules allows simultaneous capture of both alleles. If the parental allele identification is important, for example in discovering the origins of disease alleles, the dye colors are assigned to the parent of origin by means of genotyping data from at least one parent. This method enables the parent-specific identification into maternal and paternal pools of multiple different chromosomes (e.g. chr1, chr2, . . . ) simultaneously into two separate tubes, wells or compartments. In certain cases, there may be other reasons to isolate only a single chromosome at a time, for example if a chromosome or large locus on one chromosome is already associated with the disorder.

There are several distinct approaches to the PCR amplification that will be described in the next sections. In a first approach, PCR amplification is performed in a steady state process on the microfluidic chip with integrated spatially addressable thermal cycler. In another embodiment, the whole chip is thermally cycled, and in a third a chamber filled with droplets, each containing a droplet, or an emulsion or droplets, is removed and thermally cycled.

In one embodiment, the method allows simultaneous identification of maternal and paternal alleles. In this example, two distinct molecular beacons are used, with one beacon within the bounds of each primer pair for each variant interrogated. An example for the detection of a biallelic CNV is depicted in FIG. 5. In this example, the allele with the indel sequence present is reported by a green reporter. The other pair is optimized for the deletion allele and is reported by a red reporter. The longer target (for the non-deletion allele) contains sequences for both reporters, but its amplification is suppressed by making the PCR extension time sufficient for amplification of the shorter target, but insufficiently long for the longer target while in competition with the shorter target. Alternatively, if the junction site of the CNV is known to the base-pair, then one of each primer pair can be designed to span a junction site. The molecular beacon can be a hairpin oligonucleotide with a dye fluorophore at one end, and at the other end a quencher that can efficiently suppress the fluorescence of the dye, when the oligo is in the hairpin conformation. The sequence of one end of the stem and the loop is designed to be complementary to the target sequence, and the loop complementary to a small region at the other end of the oligo. When bound to the target the dye and quencher are too far apart for the dye to be quenched, but when unbound in solution the hairpin structure efficiently quenches the dye molecule. This and other equivalent amplification techniques are not new and known to those skilled in the art.

In one embodiment, a slide that contains microwells is exposed to a solution of metaphase chromosomes in such a way that the microwells are occupied by a small number of chromosomes (e.g., a single chromosome, or an average less than one), but with a density optimized for overall system performance. In some embodiments there are less than 10 chromosomes in each microwell. If only a single homologous pair of chromosomes is desired, then the extra chromosomes are inconsequential, as they only result in some amount of superfluous sequencing. In cases where multiple chromosome may exist in the same well, then it is useful to use the two-color biallelic assay so that if both of the same homologous pair of chromosomes occupies the well, the PCR will be positive for both alleles, and the well can be ignored or rejected. This approach relies on a technique for individually capturing the contents of each well. This can be by micro-pipetting or by ejecting the contents of each well into tubes, by parent of origin, so a robotic system similar to that of an LCM may be used for high throughput studies.

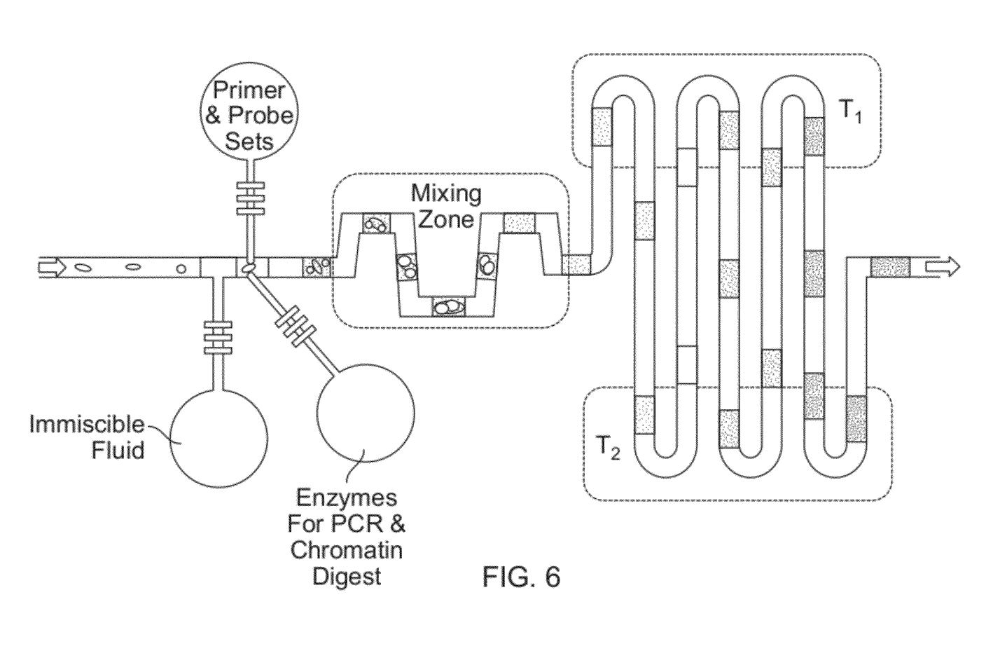

Once parent-specific primers and molecular beacons are selected, then the next step is the amplification of the variants of interest for the identification of the parent of origin of each chromosome of interest flowing through the system. In one embodiment depicted schematically in FIG. 6, chromosomes are encapsulated into droplets along with their parent specific primers and amplification reagents in cells of immiscible fluid.

The fluid flowing in from the left in FIG. 6 contains primarily intact metaphase chromosomes flowing through a microchannel in an aqueous-based buffer solution. In the embodiment shown in FIG. 6, a second fluid, one that is immiscible with the first, is injected into a channel in such a way that many of the chromosomes become individually confined within alternating plugs of fluid within the channels, where a “plug” of fluid is a continuous region of similar fluid sandwiched between regions of a dissimilar fluid immiscible with the plug. This can be most simply enabled by periodically injecting a separation fluid, without prior knowledge of the positions of the chromosomes in the channel. In this case, a small fraction of the plugs will contain a chromosome, and a substantially smaller fraction may contain multiple chromosomes. Alternatively, droplets can be created by monitoring the flow of chromosomes, either manually or with a detector, or with a vision system. In the former case, a detection system downstream determines which plug contains one or more chromosomes, and may even estimate the number of chromosomes within each plug. The latter case enables the optimization of the creation of fluid plugs such that they are most likely to contain a single chromosome. Once these plugs have been created, they can be selected downstream based on their chromosomal contents.

FIG. 6 depicts the injection of amplification reagents into the plugs containing a chromosome. These reagent fluid contains primers, molecular beacon probes, enzymes for PCR amplification of the specific CNV targets, and possibly other reagents for the digestion of residual proteins or fragmentation of the genomic bulk of the chromosome within the plug. Also depicted in the figure is an optional region of abrupt corners or baffles to assist in the mixing of the fluids within the plug in order to homogenize the various components of each plug before amplification. The plugs of fluid are subsequently continually flowed through hot and cold regions within an amplification zone on the microfluidic chip. Different regions are maintained at different temperatures enabling continuous flow PCR. With this approach PCR products can be continuously produced perhaps in synchrony with upstream and downstream processes, or perhaps on an independent timescale. Note that the number of serpentine loops from hot to cold schematically depicted in the figure is fewer than that of a practical system, which is more likely to utilize from 10 to 40 cycles of PCR, depending on the signal needed for robust detection. Additionally, three or more different temperature zones may be necessary for efficient amplification.

The output of the variant region amplification consists of plugs that fall into four distinct classes: 1. a product of a chromosome from a first parent, e.g. a red labeled amplicon; 2. the product of a chromosome of the other parent, e.g. a green labeled amplicon; 3. chromosomes from both parents, red and green labeled amplicon (yellow), and 4. no amplification product, for example if there is no chromosome or an untargeted chromosome within the plug.

In certain embodiments, the products of this amplification step span only one or several small regions of the targeted chromosomes. The remaining portions of the chromosome(s) remain within the plug in only the two copies, from the pair of sister chromatids of the metaphase chromosome.

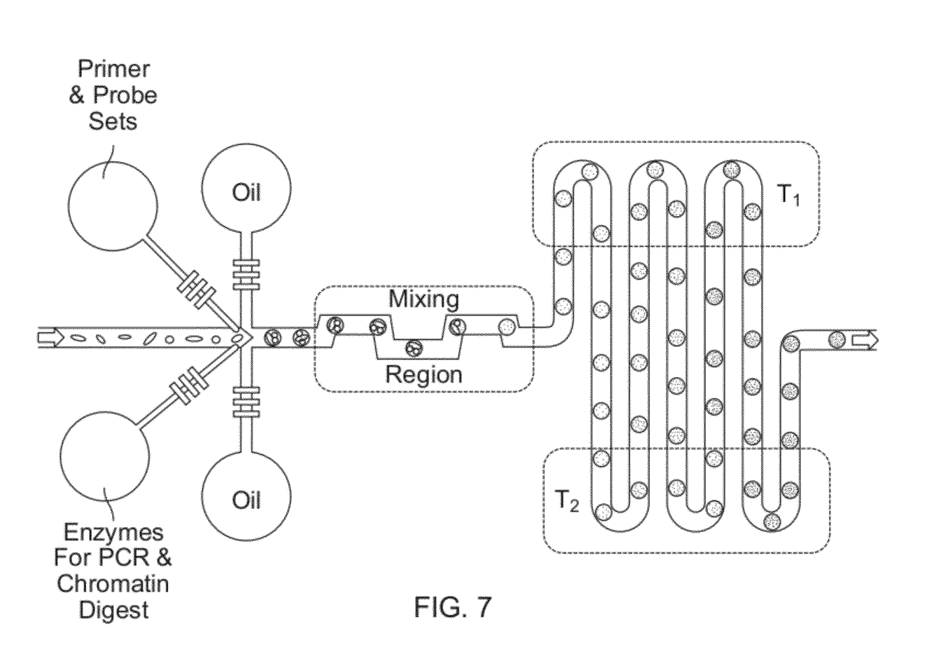

In another embodiment, rather than confining the chromosomes to plugs of immiscible fluids within a very narrow channel (as shown in FIG. 6), the chromosomes may be confined within small droplets of aqueous fluid within a non-aqueous medium e.g. oil, as shown in FIG. 7. This embodiment is similar to the embodiment with the fluid plugs, except that the size of the plugs in 2 dimensions are defined by the width and height of the channels, whereas the droplet volume is defined at the point at which the droplet breaks off from the stream. Small droplets can be formed with consistent volumes using known techniques. These droplets remain stable as they flow through the microfluidic system, and they can be amplified by on-chip continuous PCR in much the same way as the plugs described above.

In another embodiment, in order to minimize the complexity of the microfluidic chip and its holder, rather than construct a complex or large chip that can accommodate hot and cold regions, the chip itself can be constructed for the storage of many plugs. These plugs can be accommodated within long microchannels, for example, fashioned in a long serpentine pattern. Once the channel is filled with plugs of chromosome-filled plugs, the whole chip is placed into a thermal cycling oven for amplification by PCR of all plugs within the chip. In a slightly more complex embodiment, the capacity of the chip can be increased while minimizing the resistance to fluid flow by means of microvalves on the chip that can redirect the plug flow into a set of microchannels that are fabricated into the multiple channels. In this way the plugs can be stored within a multitude of distinct microchannels. Each channel may store thousands of separate plugs, perhaps even tens of thousands of plugs, with perhaps as many as millions of plugs stored within a single fluidic chip. In these embodiments, the fluids are selected such that the plugs remain immiscible over the range of temperature necessary for the PCR amplification. Similarly, the PCR primers are optimized for compatibility with the fluids used in the chip.

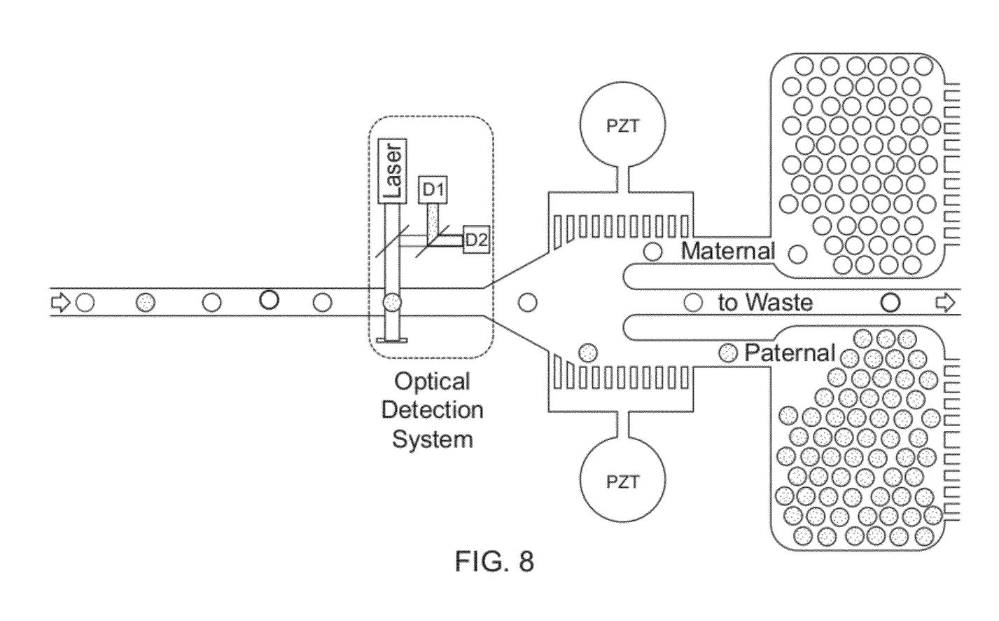

In the embodiment described previously in which droplets are amplified by PCR on chip and labeling is performed by two sets of molecular beacons, one set of beacons is labeled in a first color (e.g., red) for indicating paternal chromosomal origin and another labeled in a second color (e.g., green) indicating maternal origin. These droplets can flow through a region of the chip monitored by a detection system using a light source, including laser filter sets, such as excitation and emission filters for each dye chromophore of the molecular beacons or other fluorescently labeled reporter, and one or more detectors, as drawn schematically in FIG. 8. When the droplet flows through the detection region, it is excited by a light source, such as a laser or LED, and the emission signal from a detector is monitored to determine whether the molecular beacons within the droplet indicate its parental origin. When a beacon signal is detected, then the droplet is diverted into the appropriate storage compartment or well. The mechanism for the diversion shown in the figure is only one possible isolating embodiment, again another embodiment utilizing valves can be used. Further, in that case, instead of a 3-way diverting mechanism as shown, two serial 2-way diverters (not shown) can be used.

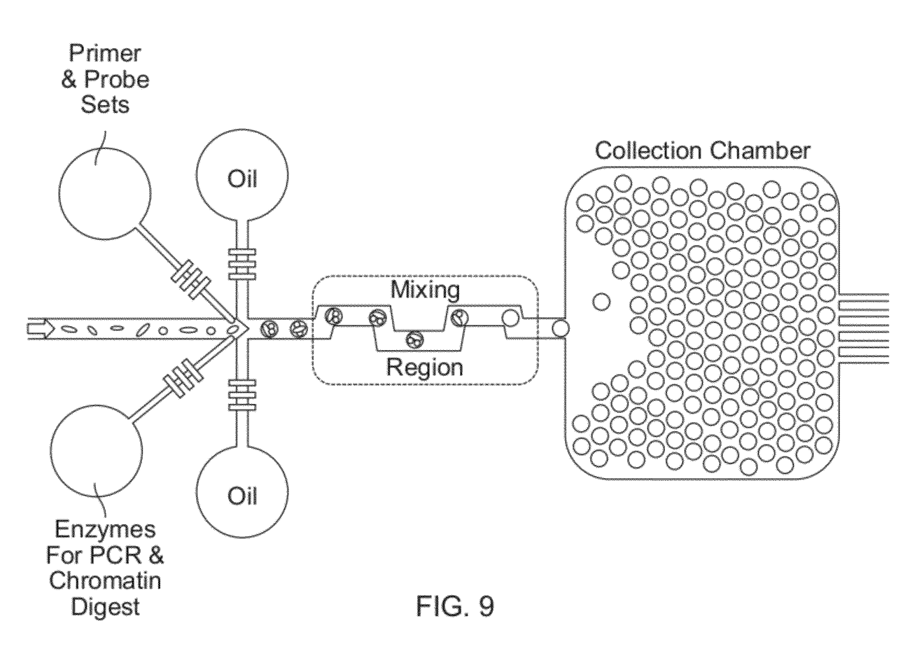

As an alternative to those embodiments involving on-chip amplification described above, a large number of droplets can be stored as an emulsion in a collection chamber, either on-chip or off-chip, as depicted in FIG. 9. The collection chamber consists of an inlet port that allows the inflow of droplets and an output filter port that allows the outflow of the fluid media without passing the droplets containing genomic material. Once collected, the fluid can either be transferred to another device or the chamber itself can be cycled in a thermal-cycling oven (not shown).

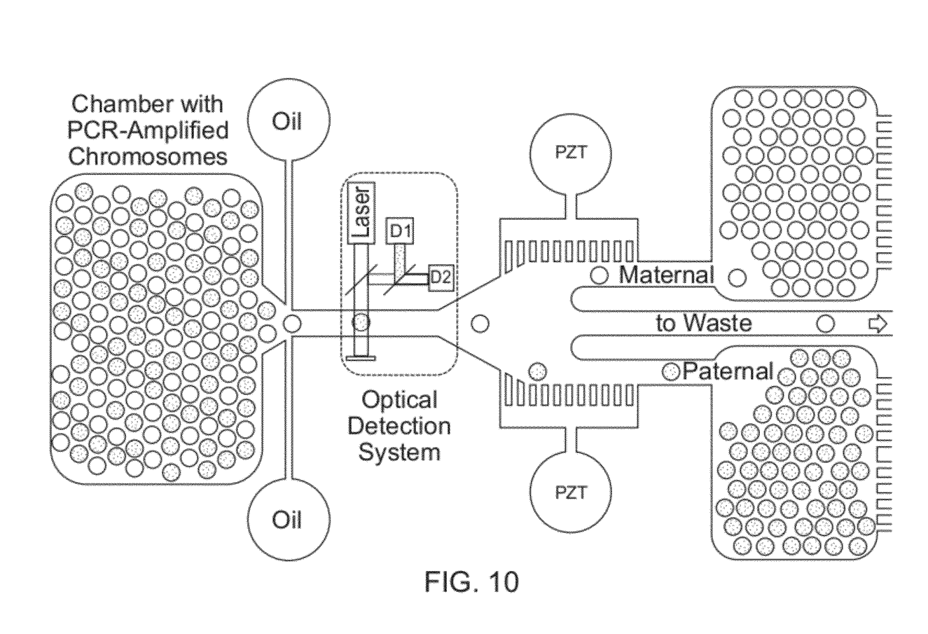

Once the emulsion has been amplified by PCR, then the droplets containing chromosomal material can be fluorescently labeled by the molecular beacons according to their parent of origin (as described above). The emulsion can then be run through a sorting mechanism, such as the one depicted in FIG. 10. The mechanism here consists of the optical detection system and sorting mechanism (both described above). Oil is injected into the channel after the port from the collection chamber in order to increase the distance between droplets so that they pass through the detection and selection region singularly. The maternal and paternal chromosomal droplets are pooled into separate collection compartments.

An alternative embodiment for the sorting of labeled genomic material, whether in droplets or in plugs, can be performed running labeled droplets through a nozzle for isolation of chromosomal material by parent of origin. A FACS is a Fluorescently Activated Cell Sorter, and these instruments, as their name suggests, are traditionally used for sorting whole cells e.g. blood cells, stem cells, etc, but they can also be used to sort other materials that can formed into a liquid droplet of the appropriate size. A FACS works by firing a droplet that may be labeled in or (or more) distinct colors along a path between two parallel electrostatic plates. As the droplet flies past a fluorescence detection system, its fluorescence signal is detected and it is classified in flight by a computer that determines into which tube the droplet should be collected. In the case of this application, the targeted tubes are those of the two parents of origin and a waste tube. If the signal for either color channel is positive, then the appropriate parental tube is targeted, if neither or both of the signal channels are detected then the droplet remains undeflected and is directed into a waste tube. Applying this approach with dual-color selection is advantageous over the same with a single color selection, especially if the probability that multiple chromosomes may occupy the same droplet is significant.

This sorting can be performed using either of two approaches. In a first approach, each droplet is encapsulated within a single larger fluid droplet that is fired by the nozzle or inkjet as a single droplet. In this case the chromosome within the droplet can remain intact. In a second approach, the droplets generated within the FACS are smaller than the volume of each microfluidic plug. In this latter case, if the chromosomes is digested and each droplet fired from the jet, each droplet contains a fraction of the chromosome and many labeled reporter probes, and each droplet is isolated according to the label indicating the parent of origin. Most of each chromosome, however will be collected by capturing numerous droplets. This approach enables high throughput collection of chromosomal material.

### Genotyping

Once the maternally-derived chromosome and/or the paternally-derived chromosome has been isolated from other chromosomes in the labeled sample on the basis of its labeling to produce an isolated maternally-derived chromosome and/or an isolated paternally-derived chromosome; the isolated chromosomes are independently genotyped. Any suitable genotyping method may be employed in this step. In one embodiment, the genotype may include determining the relative copy numbers of sequences in the maternally-derived and homologous paternally-derived chromosomes; or determining the status of SNPs in the maternally-derived and homologous paternally-derived chromosomes. In another embodiment, an epigenetic modification of an isolated chromosome may be assayed by determining the methylation status or histone modification status of the maternally-derived and homologous paternally-derived chromosomes. Methods for performing such assays are well known, and may include sequencing, array analysis, immunoprecitation of modified sequences or histone modifications, and PCR analysis, as well as a number of other techniques.

Specifically, once the parentally-derived chromosomes of haploid pools of chromosomes have been isolated, virtually any detection method can be applied to the chromosome pools, including genotyping of the SNPs, CNVs, in-dels, methylation or looking for epigenetic events in each pool independently. These methods may include (but are not limited to): sequencing by any method, microarray detection, SNP array detection, CGH array detection and methylation detection. For genotyping, haploid analysis methods should be applied rather than diploid analysis methods.

In some embodiments, once the genomic material has been separated into two collection vessels, the contents of each can be manipulated as a single sample. For example, each pool can be centrifuged to combine all the genomic material within the aqueous solution and separate it from the other oily isolation fluid. Subsequent to that separation, the mixture can be manipulated for the purpose of genome-wide amplification (if necessary) and sequencing, or other form of genotyping. As all steps of this process are automatable, this method should be high throughput allowing many thousands of chromosomes to be collected in minutes. As an example, some digital PCR methods using emulsified droplets can sort those droplets at rates of >1,000 droplets per second. The higher the copy numbers of collected material, the lesser the amplification necessary (if any) before sequencing and the lower the uncertainties in the sequences produced. Low noise will make possible not only robust calls in unique regions of the genome but also should enable allelic copy number estimates in duplicated genomic regions.

In addition, there are a number of different methods for high-throughput sequencing, including those provided by Illumina, Life Technologies and Pacific Biosciences. The samples resulting from this method should be compatible with all these technologies, and should also be compatible with target enrichment methods. Further, if distinct chromosomes are collected independently there may be some utility in ligating primers that include barcode sequences for each independent chromosome i.e. chr1, chr2 . . . chrX.

The primary preparation that should be performed before sequencing is the fragmentation and size selection of DNA in order to remove the labeled PCR primers and PCR products. For this reason, the PCR product lengths should be kept sufficiently small (<100 bp) that they can be efficiently removed from the target pool before genome-wide sequencing. Any fragment method may be applicable for the genomic DNA, including, restriction digestion, sonication and heat-shock. However, random methods, such as sonication are preferred as they reduce systematic biases against shorter sequences that would result from the deterministic fragmentation lengths produced by restriction enzymes.

The method described above allows “phased-genotyping” over the entire lengths of whole chromosomes. The method works by identifying each of the two distinct chromosomes, the maternally-derived and paternally-derived chromosomes, then by isolating those parent-specific chromosomes independently, before genotyping the subsequent haploid samples.

The method can be used to determine the number of functional gene copies of specific disease related genes for diagnoses and treatments of known monogenetic disorders. Additionally, this method will find application in elucidating the relevance for all genes on a chromosome, or across the whole genome, for application to more complex multigenic disorders.

In certain embodiments, the method may be employed, for example, to identify a difference in nucleotide sequence, a difference in copy number of a sequence, a difference in methylation or a difference in histone acetylation between a maternally-derived chromosome and a homologous paternally-derived chromosome. In particular cases, the method may be employed to identify mutations in the nucleotide sequence of the same locus in both the maternally-derived and the homologous paternally-derived chromosomes, whether the mutations are at the same position or at different positions in the maternally-derived and paternally-derived chromosomes. In one embodiment, the mutations may affect the expression of the same gene or may affect the activity of the encoded protein in both the maternally-derived and the homologous paternally-derived chromosomes.

In accordance with the above, a method of sample analysis is provided. In certain embodiments the method comprises: a) obtaining from a diploid individual a chromosomal sample that comprises maternally-derived chromosomes and homologous paternally-derived chromosomes; b) hybridizing to the chromosomal sample, in situ, a labeled nucleic acid probe that differentially hybridizes to a maternally-derived chromosome and a homologous paternally-derived chromosome, thereby providing a labeled sample in which the maternally-derived chromosome and the homologous paternally-derived chromosome are distinguishably labeled; c) isolating one or both of the maternally-derived chromosome and the paternally-derived chromosome from other chromosomes in the labeled sample on the basis of the labeling to produce an isolated maternally-derived chromosome and/or an isolated paternally-derived chromosome; and d) independently genotyping the isolated maternally-derived chromosome and/or an isolated paternally-derived chromosome.

In certain embodiments, the labeled nucleic acid probes hybridize to a copy number variation. In certain embodiments, the copy number variation is a sequence that is present in one of the maternally-derived or homologous paternally-derived chromosomes and not present in the other of the chromosomes. In certain embodiments, the nucleic acid probe is labeled with a fluorescent label. In certain embodiments, the isolating is done by flow cytometry. In certain embodiments, the isolating is done by flow cytometry of metaphase chromosomes bound to beads. In certain embodiments, the isolating is done by magnetic cytometry. In certain embodiments, the isolating is done by laser microdisection. In certain embodiments, the isolating is done by manual manipulation. In certain embodiments, the genotyping comprises determining the relative copy numbers of sequences or determining the status of SNPs in the maternally-derived and homologous paternally-derived chromosomes. In certain embodiments, the genotyping comprises determining the methylation status or histone modification status of the maternally-derived and homologous paternally-derived chromosomes. In certain embodiments, the genotyping is done by sequencing. In certain embodiments, the genotyping is done by array analysis. In certain embodiments, the genotyping is done by PCR. In certain embodiments, the genotyping identifies a difference in nucleotide sequence between the maternally-derived chromosome and the paternally-derived chromosome. In certain embodiments, the genotyping identifies mutations in the nucleotide sequence of the same locus in both the maternally-derived and the homologous paternally-derived chromosomes. In certain embodiments, the mutations affect the expression of the same gene in both the maternally-derived and the homologous paternally-derived chromosomes. In certain embodiments, the mutations are at the same position in the maternally-derived and paternally-derived chromosomes. In certain embodiments, the mutations are at different positions in the maternally-derived and paternally-derived chromosomes. In certain embodiments, the individual is a mammal. In certain embodiments, the mammal is a human.

### EXAMPLES

The following examples are put forth so as to provide those of ordinary skill in the art with a description of how to make and use some embodiments of the present invention, and are not intended to limit the scope of what the inventors regard as their invention.

### CNV-FISH Chromosomal Identification

Parent-of-origin specific identification of chromosomes has been demonstrated using oligo-FISH probes. The sample used for this demonstration is a cell-line derived from the daughter of a family trio from Yoruba, Africa. This sample consists of metaphase chromosome preparation on a glass slide using cells of a lymphoblastoid cell-line from Coriell Cell Repositories for HapMap sample GM19240. This sample was previously characterized in various publications, including Campbell et al (AJHG, 88, 317-332, 2011), Mills et al (Nature, 470: 60 2011) and Conrad et al (Nature 2010 464: 704-1). From the published data, a list of CNVs for which the genotypic states of the CNVs are known for the daughter as well as her parents was compiled. Using this information, the parental origin of several relatively large biallelic deletion-type CNVs was determined. oligoFISH probes for four of these heterozygous CNV's residing on chromosomes 1, 4, 6 and 8 were constructed.

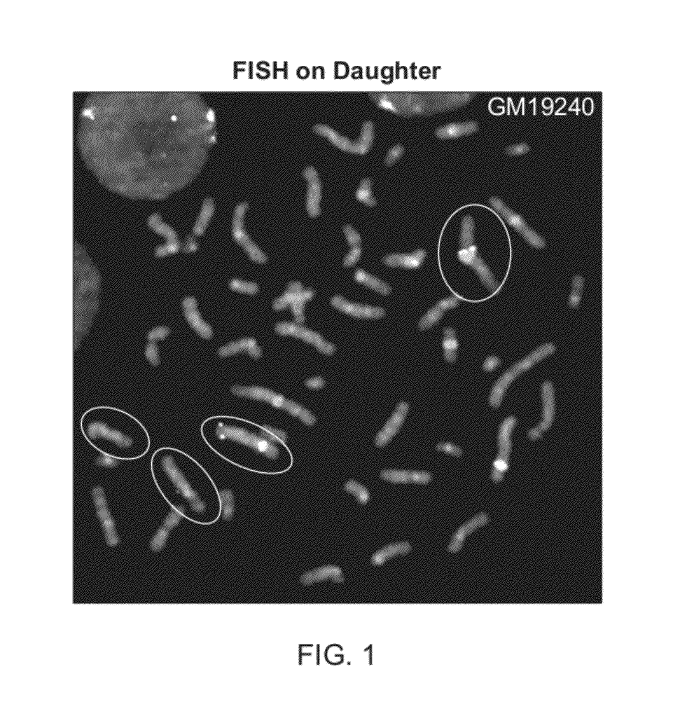

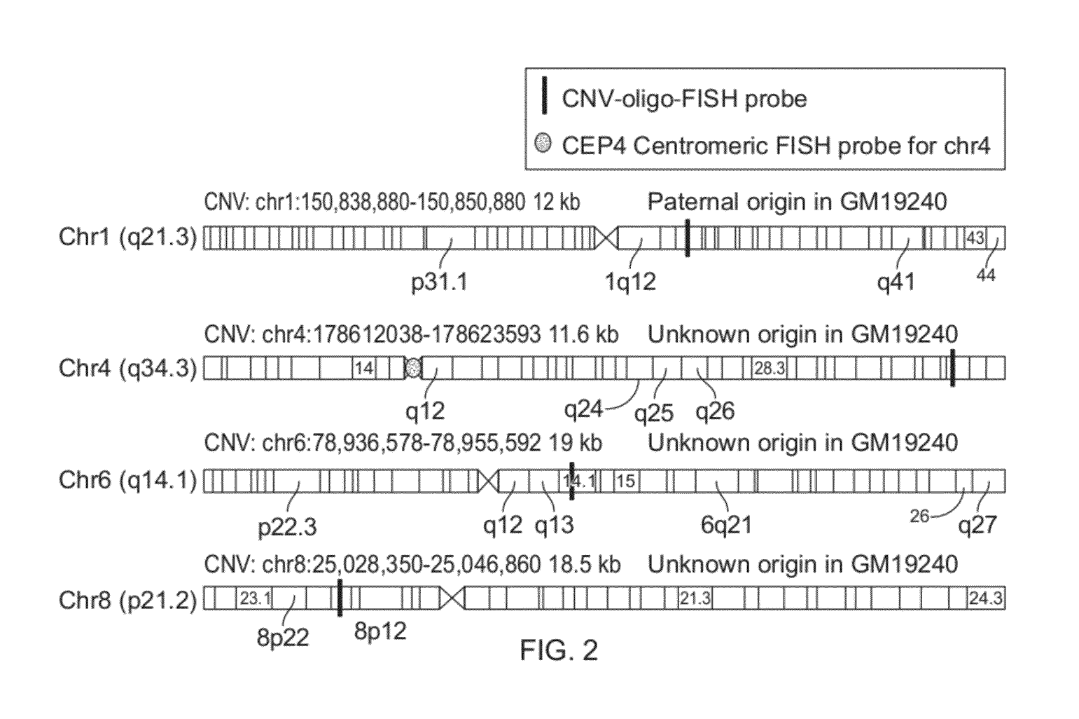

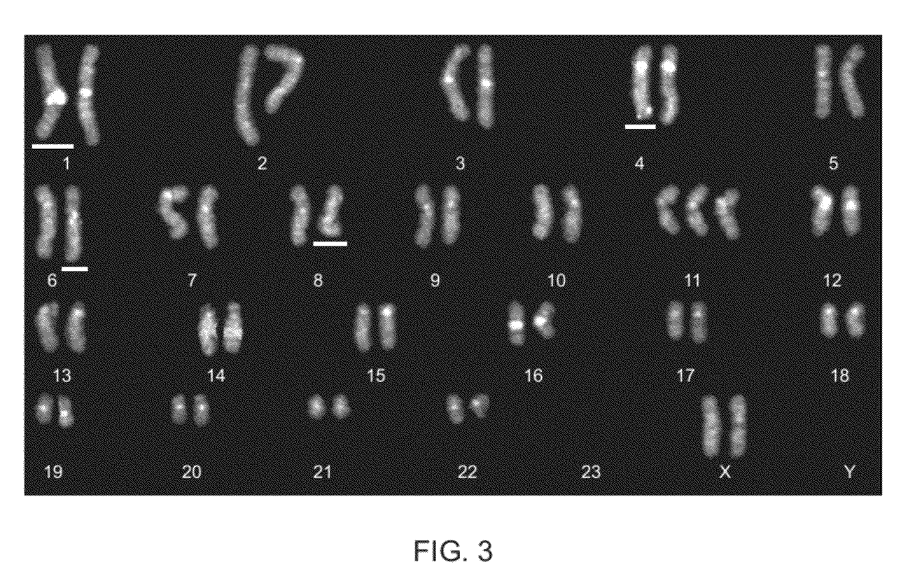

A FISH-hybridization assay was performed on a metaphase slide for the GM19240 cell line, and a chromosome spread from a single cell is shown in FIG. 1. Four oligo FISH probes and one centromeric BAC probe were used. Ideograms indicating the positions of the FISH probes on the chromosomes is depicted schematically in FIG. 2. FIG. 3 shows a reorientation of the sub-images (from FIG. 1) arranged in homologous pairs using the Smart Type software with some manual identification of the homologous chromosome pairs. The images show that one chromosome of each of the four homologous pairs (chr1, chr4, chr6 & chr8) is each marked by two fluorescent spots (one for each of the sister chromatids, as duplicated in G2-phase). The FISH markers can be used along with the genotypes of the parents to identify the parent-of-origin for each chromosome of each marked pair.

In this example, only the pair of homologous chromosomes for chromosome 4 is identified by a green centromeric BAC-FISH probe (green) in addition to the oligo-FISH probe targeting the CNV. Any diploid region on any normal chromosome that is present in both homologous can be used copies to identify both chromosomes of each homologous pair. The extraction of chromosomal material from the slide is known to those skilled in the art of chromosome laser capture microdisection. These methods are enabled by commercial laser capture microdisection systems from several manufacturers, including: Carl Zeiss, Inc., and the Arcturus™ product line of Life Technologies. Such systems can be used to extract chromosomal material from the surface of a glass slide or from a membrane slide either by “catapulting” the chromosomal material itself, by cutting the perimeter of a small portion of a polymer membrane containing a chromosome from the slide, and catapulting from a slide (Zeiss LCM) or by adhering that small region to an adhesive surface (Arcturus LCM). Such commercial systems are currently used to collect chromosomal material used for the manufacturing probes for an application known as chromosome painting.

In certain cases, while using the Zeiss LCM system, the fluorescent dyes (Cy3 and Cy5) FISH probes were quenched when exposed to air. Consequently, the fluorescence images were made in a glycerol media while each image was mapped to a position on the slide. The glycerol media was subsequently washed away and the chromosomes were stained with Giemsa which can be observed with the light microscope on the LCM. This allowed identification of the chromosomes using the positions of the spreads and the fluorescent images. This process, though done manually in this demonstration, can be automated for high throughput applications. Alternatively, the process could be accomplished either by using non-quenching dyes or by operating the LCM in an oxygen-free chamber.

1. A method of sample analysis comprising:
a) obtaining from a diploid individual a chromosomal sample that comprises maternally-derived chromosomes and homologous paternally-derived chromosomes;
b) determining the parent of origin of a first chromosome of said sample by detecting a parent-specific copy number variation relative to a second chromosome that is homologous to the first chromosome;
c) isolating said first chromosome after its parent of origin is determined; and
d) genotyping the isolated chromosome of step c).

2. The method of claim 1, wherein:
said determining comprises determining the parent of origin of a plurality of chromosomes in said sample;
said isolating comprises pooling at least two chromosomes of the same parental origin; and
said isolating comprises genotyping said at least two chromosomes of the same parental origin.

3. The method of claim 2, wherein said at least two chromosomes are the same chromosome.

4. The method of claim 2, wherein said at least two chromosomes are different chromosomes.

5. The method of claim 1, wherein said copy number variation is a nucleotide sequence that is present in one of said maternally-derived or homologous paternally-derived chromosomes and not present in the other of said chromosomes.

6. The method of claim 1, wherein said genotyping comprises sequencing at least part of the first chromosome.

7. The method of claim 1, wherein said genotyping is done by array analysis.

8. The method of claim 1, wherein said genotyping is done by PCR.

9. The method of claim 1, wherein said method comprises
hybridizing to said chromosomal sample, in situ, a labeled nucleic acid probe that differentially hybridizes to a copy number variation that distinguishes a maternally-derived chromosome and a homologous paternally-derived chromosome;
isolating said maternally-derived chromosome or said paternally-derived chromosome from other chromosomes in said sample on the basis of said labeling to produce an isolated maternally-derived chromosome or an isolated paternally-derived chromosome; and
genotyping said isolated maternally-derived chromosome or said isolated paternally-derived chromosome.

10. The method of claim 1, wherein said method comprises:
isolating the individual chromosomes;
determining the parent of origin of a chromosome by polymerase chain reaction (PCR); and
genotyping said chromosome.

11. The method of claim 10, wherein said method comprises:
depositing individual chromosomes of said sample into separate wells;
determining the parent of origin of a chromosome by polymerase chain reaction; and
genotyping said chromosome.

12. The method of claim 10, wherein said method comprises:
separating said chromosomes into discrete plugs in the flow stream of a microfluidics device;
determining the parent of origin of said first chromosome of said sample by PCR in said plug, wherein the parent of origin of said first chromosome is indicated by fluorescence;
collecting a plug containing a chromosome on the basis of its fluorescence; and
genotyping said collected first chromosome.

13. The method of claim 1, wherein said isolating comprises pooling at least ten chromosomes of the same parental origin, wherein said at least ten chromosomes are the same chromosome and said genotyping comprises subjecting the pooled sample to at least two different genotyping methods.

14. The method of claim 1, wherein said genotyping comprises determining the relative copy numbers of sequences or determining the status of SNPs in said first chromosome.

15. The method of claim 1, wherein said genotyping comprises determining the methylation status or histone modification status of said first chromosome.

16. A microfluidic device comprising:
a fluid flow path comprising an aqueous solution of metaphase chromosomes;
a reservoir of reagents connected to said fluid flow path, comprising PCR reagents for detecting the parent of origin of at least one of said chromosomes by PCR, and chromatin digestion reagents; and
a reservoir of an immiscible fluid connected to said fluid flow path via a valve, wherein said valve is controlled to produces plugs, separated from one another by said immiscible fluid, each comprising the DNA of a single metaphase chromosome and said PCR reagents.

17. The microfluidic device of claim 16, further comprising a thermocycling device to perform in plug PCR.

18. The microfluidic device of claim 16, further comprising a plug collection chamber for collecting said plugs.

19. The microfluidic device of claim 16, further comprising a gating mechanism for separating said plugs based on fluorescence.

20. The microfluidic device of claim 19, wherein said gating mechanism comprises passing said plugs through a nozzle to produce a stream of droplets, and deflecting said droplets by applying a charge.
